(19) United States
(12) Patent Application Publication
(10) Pub. No.: US 2007/0276139 A1
(43) Pub. Date: Nov. 29, 2007

(54) Substituted Pixyl Protecting Groups for Oligonucleotide Synthesis

(76) Inventors: Quanlai Song, Carlsbad, CA (US); Sak Khammungkhune, San Diego, CA (US); Bruce S. Ross, Carlsbad, CA (US); Richard H. Griffey, Vista, CA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 10/597,808

(22) PCT Filed: Feb. 10, 2005
(86) PCT No.: PCT/US05/04875

§ 371 Date: Mar. 20, 2007

Related U.S. Application Data
(60) Provisional application No. 60/543,234, filed on Feb. 10, 2004.
(60) Provisional application No. 60/568,587, filed on May 5, 2004.
(60) Provisional application No. 60/608,522, filed on Sep. 8, 2004.

(51) Int. Cl. C07D 311/82; C07D 335/12; C07H 19/06; C07H 21/00

(52) U.S. Cl. 544/243; 544/317; 549/223; 549/27

(57) ABSTRACT

The present invention describes an improved hydroxyl protecting group of formula (1), wherein R² and R⁷ are specified substituents and Q is O, S, NR¹⁰ or N(C═O)R¹⁰.

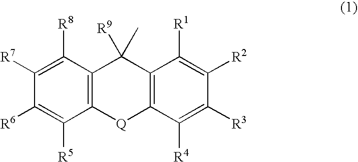

### FIELD OF THE INVENTION

The present invention describes an improved hydroxyl protecting group, and methods of using said reagent in oligonucleotide synthesis. The present invention is directed to the field of manufacture of reagents, nucleoside derivatives, nucleoside phosphoroamidites and oligonucleotide derivatives thereof, as well as methods of using said pixylating reagents and derivatives.

### BACKGROUND OF THE INVENTION

Oligonucleotides are used in various biological and biochemical applications. Presently, oligonucleotides are used as primers and probes for polymerase chain reaction (PCR), as antisense agents used in target validation, drug discovery and development, as ribozymes, as aptamers, and as general stimulators of the immune system. As oligonucleotides have become widely used in diagnostic applications and increasingly acceptable as therapeutic compounds, the need for producing greater sized batches, and greater numbers of small-sized batches, has increased at pace. Additionally, there has been an increasing emphasis on reducing the costs of oligonucleotide synthesis, and on improving the purity and increasing the yield of oligonucleotide products.

The manufacture of oligonucleotides is a multi-step process, as represented in scheme 1 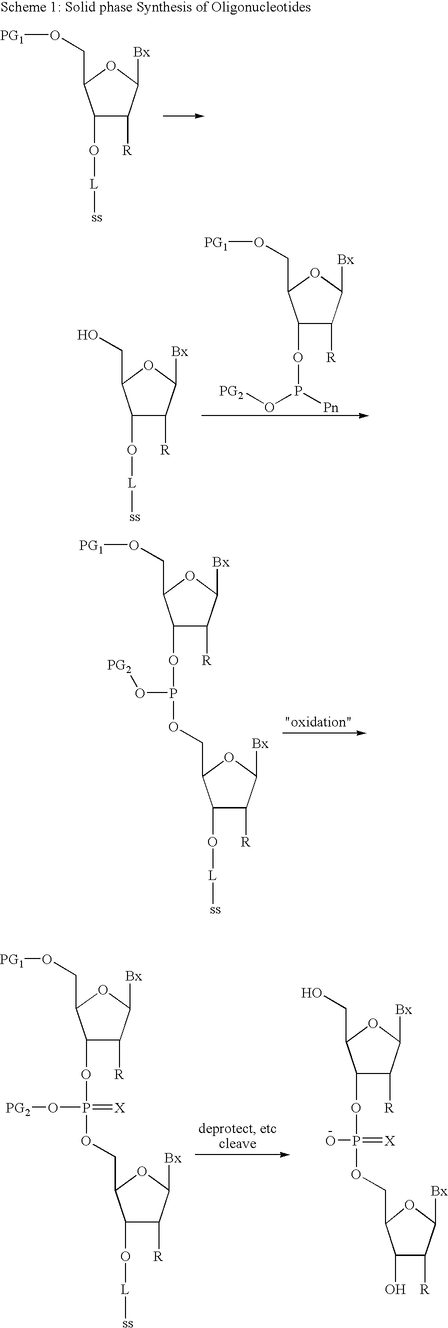
wherein, ss is a solid support medium, L is a linking moiety, Pn is as defined below, R is H, OH or a 2′ sugar substituent, each Bx is independently a nucleobase, X is O or S, PG, is a hydroxy protecting group, and PG₂ is a phosphorous protecting group.

Oligonucleotide manufacture may be divided into two distinct operations: solid-phase synthesis using phosphoramidite chemistry followed by downstream processing. In the first operation, a fully protected oligonucleotide is assembled stepwise from the 3′- to the 5′-terminus by repetition of a four-reaction elongation cycle (5′-OH deprotection, coupling, oxidation (or sulfurization to generate a phosphorothioate) and capping) without isolation of intermediates. In the second operation, phosphorous deprotection, cleavage from the support, purification and isolation steps are performed, to afford an oligonucleotide. Typically, the terminal 5′-hydroxy protecting group is not removed from the oligonucleotide prior to cleavage from resin as it provides a hydrophobic handle required for reverse phase (RP)-HPLC purification. After RP-HPLC, and oligonucleotide isolation, the protecting group is then removed under acid conditions. Thus, there is a need for high-yielding, economical and robust methods for commercial scale production of high quality oligonucleotides.

Typical methodologies for making oligonucleotides have not fundamentally changed since the development of the dimethoxytrityl (DMT) group for protection of the 5′-hydroxy group (PG₁), and the cyanoethyl phosphorous protecting group (PG₂) by Caruthers and Koster respectively. While the coupling chemistry for oligonucleotide synthesis is relatively robust and reliable, it does suffer some drawbacks. For example, alternative chemistry using 5′-silyl protecting groups has been developed by Scaringe et al. for the preparation of RNA. However, this 5′-silyl protecting strategy is incompatible with the synthesis of phosphorothioate oligonucleotides. For phosphoramidites with bulky 2′-substituents, such as methoxyethyl (MOE), the coupling efficiency to free 5′-OH residues on solid support is diminished. The lower yields are likely due to steric hindrance in the approach of the activated phosphoroamidite to the support-bound 5′-OH, but also may result from incomplete removal of the 5′-DMT group in the previous synthesis cycle. Slow deprotection kinetics for removal of 5′-DMT groups during the oligomerization process, especially from sequences that end in T, have been documented. Also, removal of the final 5′-terminal DMT group (performed after HPLC purification) from such sequences often require 4-10 times longer contact time with acid. Use of stronger acids to remove the DMT group introduces additional impurities, during synthesis or after final purification of the complete oligonucleotide, often results in hydrolysis of purine bases from the sugar phosphate backbone, particularly from deoxynucleotide residues. Given the first-order kinetics of DMT removal and the similarity in the pKa values of adenine, guanine and DMT groups, complete removal of DMT often generates some apurinic sites in the final product.

Thus, there is a need for an improved protecting group that can be removed by acids having higher pKa's than the acids required for removal of DMT, and under conditions that cause less depurination than those conditions required to remove DMT, and can also act as a suitable hydrophobic handle during reverse phase high performance liquid chromatography. There is a need for a reagent capable of introducing such an improved protecting group, a method of introducing such a reagent, and an economical method of making such a reagent.

### SUMMARY OF THE INVENTION

The present invention describes an improved hydroxyl protecting group, and methods of using said reagent in oligonucleotide synthesis. The present invention is directed to the field of manufacture of reagents, nucleoside derivatives, nucleoside phosphoroamidites and oligonucleotide derivatives thereof.

In particular compounds of formula I are described:

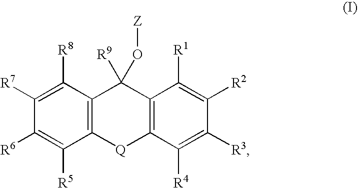

wherein:

R¹, R³, R⁴, R⁵, R⁶ and R⁸ are each, independently, H or alkyl or substituted alkyl;

R² and R⁷ are each, independently, alkyl, substituted alkyl, alkenyl, substituted alkenyl, alkynyl, substituted alkynyl, aryl, substituted aryl, hydroxyl, halo, cyano, azido, nitro, —C(═O)O—R¹⁰, —O—C(═O)—R¹⁰, —C(═O)N(R¹⁰)R¹¹, —N(R¹⁰)C(═O)R¹¹, —N(R¹⁰)R¹¹, —O—R¹⁰, or —S—R¹⁰;

or two or more groups R¹-R⁸, together with the ring carbons to which they are attached, combine to form a cyclic moiety selected from substituted or unsubstituted alicyclic, substituted or unsubstituted heterocyclic, substituted or unsubstituted aromatic, or substituted or unsubstituted heteroaromatic;

R⁹ is alkyl, substituted alkyl, alkenyl, substituted alkenyl, alkynyl, substituted alkynyl, aryl or substituted aryl;

R¹⁰ is H or alkyl;

R¹¹ is H or alkyl;

Z is a deoxy residue of a protected compound selected from a nucleoside, a nucleotide, a solid support-bound nucleotide, a nucleotide phosphoroamidite, an oligonucleotide, an oligonucleotide blockmer, or a solid support-bound oligonucleotide; and

Q is O, S, NR¹⁰, N(C═O)R¹⁰.

In some embodiments of the invention R¹, R³, R⁴, R⁵, R⁶ and R⁸ are each H. In other embodiments, the R¹, R³, R⁴, R⁵, R⁶ and R⁸ are each H, and R² and R⁷ are selected from alkyl or substituted alkyl. In yet other embodiments, any one of the protected compounds selected from a nucleoside, a nucleotide, a solid support-bound nucleotide, a nucleotide phosphoroamidite, an oligonucleotide, an oligonucleotide blockmer, or a solid support-bound oligonucleotide comprise at least one modified sugar, a 2′-substituent, or a conjugate group. In a preferred embodiment the 2′-substituent is selected from fluoro, alkoxy, substituted alkoxy, or OPR, wherein PR is a 2′-protecting group. In a further preferred embodiment the 2′-substituent is selected from fluoro, OCH₃, OCH₂CH₂OCH₃, or OCH₂CH₂ON(CH₃)₂. In another preferred embodiment, the 2′-substitutent is OPR., wherein OPR is selected from CPEP, ACE, TOM, TBDMS, or Fpmp. In another embodiment the modified sugar is a locked nucleic acid, or a 4′-thio nucleic acid. In a further embodiment the conjugate group comprises a lipophilic moiety. In a preferred embodiment, the lipophilic moiety is selected from a cholesterol moiety or a polyethylene glycol moiety.

Other aspects of the invention describe compounds of formula (II):

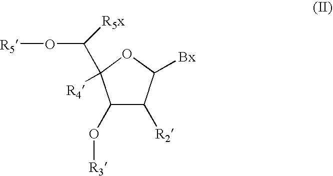

wherein

Bx is an optionally protected heterocyclic base moiety;

one of R₃′ or R₅′ is Px, wherein Px is a hydroxyl protecting group of formula I, according to claim 1, and the other is selected from:
—P(Pg)(Pn), where Pg is a phosphorus protecting group and Pn is —N(RN1)(RN2), wherein each of RN1 and RN2 is independently selected from hydrogen, substituted or unsubstituted aliphatic, substituted or unsubstituted alicyclic, substituted or unsubstituted aromatic, or substituted or unsubstituted heteroaromatic, or RN1 and RN2 are taken together with the nitrogen atom to which they are attached to form a cyclic moiety selected from substituted or unsubstituted heterocyclic;
-L-ss, where L is a linking moiety and ss is a solid support;
an H-phosphonate moiety; or
a nucleic acid moiety selected from a nucleoside, a nucleotide, a solid support-bound nucleotide, a nucleotide phosphoroamidite, an oligonucleotide, an oligonucleotide blockmer, or a solid support-bound oligonucleotide;

R₂′ is independently selected from OH, alkoxy, substituted alkoxy, halogen, OPR, where PR is a 2′-protecting group, or a nucleic acid moiety selected from a nucleoside, a nucleotide, a solid support-bound nucleotide, a nucleotide phosphoroamidite, an oligonucleotide, an oligonucleotide blockmer, or a solid support-bound oligonucleotide;

R₄′ is H or R₄′ and R₂′ are taken together to be —(CH₂)ₙ—Y—, where n is 1 or 2 and Y is selected from —O—, —S—, or —N(RN3)-, wherein RN3 is selected from H or substituted or unsubstituted aliphatic; and

R₅x is selected from H or substituted or unsubstituted alkyl.

In an alternate embodiment of the present invention are phosphoroamidites of formula II, wherein R₅′ is a 5′-protecting group, R₃′ is —P(Pg)(N(CH₂CH₃)₂), and R₂′ is —O—CPEP.

In some embodiments of the invention, R₅′ is Px and R₃′ is —P(Pg)(Pn). In preferred embodiments, Pg is —O(CH₂)₂CN and Pn is —N(CH(CH₃)₂)₂. In other embodiments R₂′ is OPR. In preferred embodiments, PR is selected from Px, CPEP, ACE, TOM, TBDMS, or Fpmp, and most preferably PR is CPEP

In yet other embodiments, R₅′ is Px and R₃′ is a nucleic acid moiety selected from a nucleoside, a nucleotide, a solid support-bound nucleotide, a nucleotide phosphoroamidite, an oligonucleotide, an oligonucleotide blockmer, or a solid support-bound oligonucleotide. Conversely, in certain other embodiments, R₃′ is Px and R₅′ is a nucleic acid moiety selected from a nucleoside, a nucleotide, a solid support-bound nucleotide, a nucleotide phosphoroamidite, an oligonucleotide, an oligonucleotide blockmer, or a solid support-bound oligonucleotide.

In preferred embodiments, any one of said nucleic acid moieties comprises a modified sugar, a 2′substituent, or a conjugate group.

Further aspects of the invention describe methods of synthesizing compounds of formula I, comprising the steps of:
providing a free hydroxyl of a compound selected from a nucleoside, a nucleotide, a nucleotide phosphoramidite, an oligonucleotide, an oligonucleotide blockmer or a solid support-bound oligonucleotide; and
reacting said compound with a protecting group of formula (III): 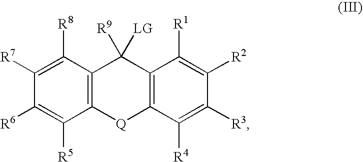
   wherein

R¹, R³, R⁴, R⁵, R⁶ and R⁸ are each, independently, H or alkyl or substituted alkyl;

R² and R⁷ are each, independently, alkyl, substituted alkyl, alkenyl, substituted alkenyl, alkynyl, substituted alkynyl, aryl, substituted aryl, hydroxyl, halo, cyano, azido, nitro, —C(═O)O—R¹⁰, —O—C(═O)—R¹⁰, —C(═O)N(R¹⁰)R¹¹, —N(R¹⁰)C(═O)R¹¹, —N(R¹⁰)R¹¹, —O—R¹⁰, or —S—R¹⁰;

or two or more groups R¹-R⁸, together with the ring carbons to which they are bonded, combine to form a cyclic moiety selected from substituted or unsubstituted alicyclic, substituted or unsubstituted heterocyclic, substituted or unsubstituted aromatic, or substituted or unsubstituted heteroaromatic;

R⁹ is alkyl, substituted alkyl, alkenyl, substituted alkenyl, alkynyl, substituted alkynyl, aryl or substituted aryl;

R¹⁰ is H or alkyl;

R¹¹ is H or alkyl;

LG is a leaving group; and

Q is O, S, NR¹⁰, N(C═O)R¹⁰.

In preferred embodiments the leaving group, LG, is chloro. In other preferred embodiments, R¹, R³, R⁴, R⁵, R⁶ and R⁸ are each H.

Additional embodiments of the present invention are methods of making any one of the compounds of formulae I or II via any synthetic method delineated herein. In yet a further embodiment is a method of synthesizing oligonucleotides either on solid support or in solution using any one of the compound of formulae I or II.

### DETAILED DESCRIPTION OF THE INVENTION

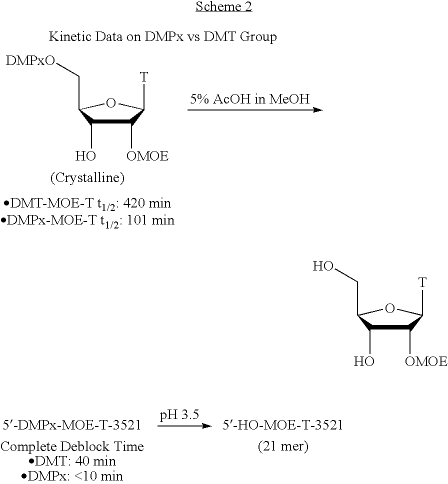

The electronic properties and pKa of the pixyl groups of the present invention can be modulated through substitution of electron-donating or electron-withdrawing substituents on the phenyl or xanthyl rings. In a preferred embodiment of the present invention, the pKa of the 5′-pixyl group is matched with the pKa of the acid chosen to effect the deprotection. Preferably, the pKa of the acid is higher than the pKa of the deoxypurine nucleobases. It is demonstrated that the kinetics of removal for an electron-donating dimethylpixyl (DMPx) group is significantly faster than a dimethoxytrityl (DMT) group. For example, the 5′-dimethylpixyl-2′-methoxyethylribothymidine (1) has a half-life of only 101 minutes upon treatment with 5% acetic acid in methanol (Scheme 2). The corresponding 5′-dimethoxytrityl compound (2) has a half-life of 420 minutes under the same conditions, as monitored using proton NMR. The deprotection time for removal of the 5′-DMPx from a 2′-methoxyethoxy(MOE)-T residue at the 5′-terminus of a synthetic oligonucleotide to be 10 minutes. The time for removal of the corresponding 5′-DMT group is 40 minutes as monitored with reverse-phase HPLC. Therefore, another embodiment of the invention is a method of removing the pixyl groups from the compounds of formula I, with an acid selected from: acetic acid, dibutylphosphoric acid, 2,2-dichloropropionic acid, dichloroacetic acid, tetrazole, salicylic acid, α-chlororbutyric acid, butyric acid, chloroacetic acid, formic acid, hexanoic acid, heptanoic acid, benzoic acid, cyanoacetic acid, pyruvic acid, acetoacetic acid, methoxyacetic acid, levulinic acid, methylthioacetic acid, pivalic acid, stearic acid, oleic acid, palmitic acid, myristic acid, malonic acid, succinic acid, adipic acid, glutaric acid, lactic acid, citric acid, malic acid, pyruvic acid, α-chlororcaproic acid, or α-methylsuccinic acid. In an preferred embodiment, DMPx protecting groups are removed with a solution comprising less than 50% acetic acid in a polar solvent.

The stability of a pixyl analog to acids is determined by the electronic stability of its cation, which is determined by the substituents on the xanthyl and aryl moieties. As a general rule, electron-donating groups make the cation more labile and electron-withdrawing groups make it more stable. A variety of pixyl analogs with different stability and crystallinity can be readily synthesized by the new synthetic route. In addition, the relative affinity of the cation or its alcohol for solid supports such as styrene may be adjusted by including hydrophilic substituents on the ring. An appropriately more labile protecting group at the 5′-end of a nucleoside and an oligonucleotide makes it possible to carry out deprotection on and after solid phase under milder conditions and minimize the depurination. For instance, 2,2-dichloropropionic acid can be used instead of dichloroacetic acid and the final deprotection can be performed at higher pHs. It also makes deprotection on solid phase more complete and decreases the formation of (n−1)mer without sacrificing the stability of monomers and increasing the longer impurity formation. An additional advantage of pixyls is the crystallinity of protected nucleosides. These favorable physical properties can simplify the purification procedures at the monomer stages and make monomers purer and subsequently oligonucleotides purer in the end.

The pixyl group has a hydrophobic character similar to the dimethoxytrityl group that makes it a suitable chromatography tag for separation of full-length oligonucleotides from untagged failure sequences. The substituted pixyl groups can be prepared in high yield from the corresponding biaryl ether and the trichloromethylphenyl group via treatment with zinc chloride and phosphorous oxychloride.

In a preferred synthetic route, the substituted pixyl chloride can be prepared in 90% yield from the appropriately substituted phenyl ether and an aromatic carboxylic acid (Scheme 2). The substituted pixyl nucleosides of the present invention are crystalline compounds, which facilitates their purification without chromatography.

As previously demonstrated in U.S. Pat. No. 6,506,894, a convergent, solution phase synthesis of DNA via 3-6mer blocks (“blockmers”) of 5′-protected, 3′-H-phosphonate monomers are likely to be the most efficient and scalable method to produce commercial quantities of therapeutic oligonucleotides. This method can be further improved by combining a 5′ substituted pixyl protecting group of the present invention. This new combination can be used to incorporate various 2′-substituted nucleotides, including, but not limited to, 2′-deoxy, 2′alkoxy, 2′ substituted alkoxy, 2′-deoxy-2′-halo (e.g., fluoro), and 2′-protected (e.g., Cpep or tBDMS) nucleotides, into oligonucleotide products.

The use of substituted pixyl nucleoside derivatives over dimethoxytrityl (DMT) ones allow for certain advantages:
(1) Pixyl derivatives of the present invention are more amenable to purification via crystallization than DMT derivatives, thereby allowing for more facile purification of 5′-protected nucleotide phosphoroamidites and oligonucleotide blockmers. Currently, each DMT derivative from monomers to each length of oligonucleotide blockmer must be purified by silica gel chromatography which limits the scale at which DMT monomers and oligonucleotide blockmers may be synthesized. Purification by crystallization is often superior, especially at production scale, because of decreased costs of purification.
(2) The pixyl derivatives of the present invention may also be optimized to have a particular acid stability for the base and sugar components in use. Both the pixyls and DMT are removed with acid and therefore leaves the rest of the oligonucleotide vulnerable to degradation (i.e., deoxyadenosine and deoxyguanonsine will depurinate and acid sensitive RNA protecting groups such as Cpep will also degrade if there are any traces of water present). Solution phase oligonucleotide synthesis requires that longer acid exposure times to effect efficient and complete deprotection thereby exacerbating the problem of degradation. The more acid-sensitive substituted pixyls of the present invention will allow for less acid exposure during solid and solution phase oligonucleotide synthesis and therefore decrease acid caused degradation.
(3) The substituted pixyl cations of the present invention can be scavenged more efficiently than the DMT cation. As noted above, solution phase synthesis requires longer exposure to acidic deprotection conditions and requires a means by which to clear the resulting protecting group cation from the resulting solution. To minimize the reverse reaction, the cation can be scavenged and trapped with a nucleophile which would compete with the 5′-hydroxyl. Reese has shown that adding pyrrole or triethylsilane efficiently traps pixyl cations and thus allows for even less exposure to acid.
   Definitions
   General Chemistry

The term “alkyl,” as used herein, refers to saturated, straight chain or branched hydrocarbon moieties containing up to twenty four carbon atoms. The terms “C₁-C₆ alkyl” and “C₁-C₁₂ alkyl,” as used herein, refer to saturated, straight chain or branched hydrocarbon moieties containing one to six carbon atoms and one to twelve carbon atoms respectively. Examples of alkyl groups include, but are not limited to, methyl, ethyl, propyl, isopropyl, n-hexyl, octyl, decyl, dodecyl and the like.

An “aliphatic group,” as used herein, is an acyclic, non-aromatic moiety that may contain any combination of carbon atoms, hydrogen atoms, halogen atoms, oxygen, nitrogen, sulfur, phosphorus or other atoms, and optionally contain one or more units of unsaturation, e.g., double and/or triple bonds. An aliphatic group may be straight chained, or branched and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted.

Suitable substituents of the present invention include, but are not limited to, F, Cl, Br, I, OH, protected hydroxy, aliphatic ethers, aromatic ethers, oxo, azido, imino, oximino, NO₂, CN, COOH, C₁-C₁₂ alkyl optionally substituted, C₂-C₁₂ alkenyl optionally substituted, C₂-C₁₂ alkynyl optionally substituted, NH₂, protected amino, N(H)C₁-C₁₂ alkyl, N(H)C₂-C₁₂ alkenyl, N(H)C₂-C₁₂ alkynyl, N(H)C₃-C₁₂ cycloalkyl, N(H) aryl, N(H) heteroaryl, N(H) heterocycloalkyl, dialkylamino, diarylamino, diheteroarylamino, OC₁-C₁₂ alkyl, OC₂-C₁₂ alkenyl, OC₂-C₁₋₂ alkynyl, OC₃-C₁₂ cycloalkyl, 0 aryl, 0 heteroaryl, 0 heterocycloalkyl, C(O)C₁-C₁₂ alkyl, C(O)C₂-C₁₂ alkenyl, C(O)C₂-C₁₂ alkynyl, C(O)C₃-C₁₂ cycloalkyl, C(O) aryl, C(O) heteroaryl, C(O) heterocycloalkyl, C(O)NH₂, C(O)N(H)C₁-C₁₂ alkyl, C(O)N(H)C₂-C₁₂ alkenyl, C(O)N(H)C₂-C₁₂ alkynyl, C(O)N(H)C₃-C₁₂ cycloalkyl, C(O)N(H) aryl, C(O)N(H) heteroaryl, C(O)N(H) heterocycloalkyl, C(O)OC₁-C₁₂ alkyl, C(O)OC₂-C₁₂ alkenyl, C(O)OC₂-C₁₂ alkynyl, C(O)OC₃-C₁₂ cycloalkyl, C(O)O aryl, C(O)O heteroaryl, C(O)O heterocycloalkyl, OC(O)NH₂, OC(O)N(H)C₁-C₁₂ alkyl, OC(O)N(H)C₂-C₁₂ alkenyl, OC(O)N(H)C₂-C₁₂ alkynyl, OC(O)N(H)C₃-C₁₂ cycloalkyl, OC(O)N(H) aryl, OC(O)N(H) heteroaryl, OC(O)N(H) heterocycloalkyl, N(H)C(O)C₁-C₁₂ alkyl, N(H)C(O)C₂-C₁₂ alkenyl, N(H)C(O)C₂-C₁₂ alkynyl, N(H)C(O)C₃-C₁₂ cycloalkyl, N(H)C(O) aryl, N(H)C(O) heteroaryl, N(H)C(O) heterocycloalkyl, N(H)C(O)OC₁-C₁₋₂ alkyl, N(H)C(O)OC₂-C₁₂ alkenyl, N(H)C(O)OC₂-C₁₂ alkynyl, N(H)C(O)OC₃-C₁₂ cycloalkyl, N(H)C(O)O aryl, N(H)C(O)O heteroaryl, N(H)C(O)O heterocycloalkyl, N(H)C(O)NH₂, N(H)C(O)N(H)C₁-C₁₂ alkyl, N(H)C(O)N(H)C₂-C₁₂ alkenyl, N(H)C(O)N(H)C₂-C₁₂ alkynyl, N(H)C(O)N(H)C₃-C₁₂ cycloalkyl, N(H)C(O)N(H) aryl, N(H)C(O)N(H) heteroaryl, N(H)C(O)N(H) heterocycloalkyl, N(H)C(S)NH₂, N(H)C(S)N(H)C₁-C₁₂ alkyl, N(H)C(S)N(H)C₂-C₁₂ alkenyl, N(H)C(S)N(H)C₂-C₁₂ alkynyl, N(H)C(S)N(H)C₃-C₁₂ cycloalkyl, N(H)C(S)N(H) aryl, N(H)C(S)N(H) heteroaryl, N(H)C(S)N(H) heterocycloalkyl, N(H)C(NH)NH₂, N(H)C(NH)N(H)C₁-C₁₂ alkyl, N(H)C(NH)N(H)C₂-C₁₂ alkenyl, N(H)C(NH)N(H)C₂-C₁₂ alkynyl, N(H)C(NH)N(H)C₃-C₁₂ cycloalkyl, N(H)C(NH)N(H) aryl, N(H)C(NH)N(H) heteroaryl, N(H)C(NH)N(H) heterocycloalkyl, N(H)C(NH)C₁-C₁₂ alkyl, N(H)C(NH)C₂-C₁₂ alkenyl, N(H)C(NH)C₂-C₁₂ alkynyl, N(H)C(NH)C₃-C₁₂ cycloalkyl, N(H)C(NH) aryl, N(H)C(NH) heteroaryl, N(H)C(NH) heterocycloalkyl, C(NH)NH₂, C(NH)N(H)C₁-C₁₂ alkyl, C(NH)N(H)C₂-C₁₂ alkenyl, C(NH)N(H)C₂-C₁₂ alkynyl, C(NH)N(H)C₃-C₁₂ cycloalkyl, C(NH)N(H) aryl, C(NH)N(H) heteroaryl, C(NH)N(H) heterocycloalkyl, S(O)C₁-C₁₂ alkyl, S(O)C₂-C₁₂ alkenyl, S(O)C₂-C₁₂ alkynyl, S(O)C₃-C₁₂ cycloalkyl, S(O) aryl, S(O) heteroaryl, S(O) heterocycloalkyl, SO₂NH₂, SO₂N(H)C₁-C₁₂ alkyl, SO₂N(H)C₂-C₁₂ alkenyl, SO₂N(H)C₂-C₁₂ alkynyl, SO₂N(H)C₃-C₁₂ cycloalkyl, SO₂N(H) aryl, SO₂N(H) heteroaryl, SO₂N(H) heterocycloalkyl, N(H)SO₂—C₁-C₁₂ alkyl, N(H)SO₂—C₂-C₁₂ alkenyl, N(H)SO₂—C₂-C₁₂ alkynyl, N(H)SO₂—C₃-C₁₂ cycloalkyl, N(H)SO₂ aryl, N(H)SO₂ heteroaryl, N(H)SO₂ heterocycloalkyl, CH₂NH₂, CH₂SO₂CH₃, aryl, arylalkyl, heteroaryl, heteroarylalkyl, heterocycloalkyl, C₃-C₁₂ cycloalkyl, polyalkoxyalkyl, polyalkoxy, methoxymethoxy, methoxyethoxy, SH, SC₁-C₁₋₂ alkyl, SC₂-C₁₂ alkenyl, SC₂-C₁₂ alkynyl, SC₃-C₁₂ cycloalkyl, S aryl, S heteroaryl, S heterocycloalkyl, or methylthiomethyl. It is understood that the aryls, heteroaryls, alkyls and the like can be further substituted.

The term “alkenyl,” as used herein, refers to a straight chain or branched hydrocarbon moiety containing up to twenty four carbon atoms having at least one carbon-carbon double bond. The terms “C₂-C₆ alkenyl” and “C₂-C₁₂ alkenyl,” as used herein, refer to straight chain or branched hydrocarbon moieties containing two to six carbon atoms and two to twelve carbon atoms respectively and having at least one carbon-carbon double bond. Examples of alkenyl groups include, but are not limited to, ethenyl, propenyl, butenyl, 1-methyl-2-buten-1-yl, alkadienes and the like.

The term “substituted alkenyl,” as used herein, refers to an “alkenyl” or “C₂-C₁₂ alkenyl” or “C₂-C₆ alkenyl,” group as previously defined, substituted by one, two, three or more substituents.

The term “alkynyl,” as used herein, refers to a straight chain or branched hydrocarbon moiety containing up to twenty four carbon atoms and having at least one carbon-carbon triple bond. The terms “C₂-C₆ alkynyl” and “C₂-C₁₂ alkynyl,” as used herein, refer to straight chain or branched hydrocarbon moieties containing two to six carbon atoms and two to twelve carbon atoms respectively and having at least one carbon-carbon triple bond. Examples of alkynyl groups include, but are not limited to, ethynyl, 1-propynyl, 1-butynyl, and the like.

The term “substituted alkynyl,” as used herein, refers to an “alkynyl” or “C₂-C₆ alkynyl” or “C₂-C₁₂ alkynyl,” group as previously defined, substituted by one, two, three or more substituents.

The term “alkoxy,” as used herein, refers to an aliphatic group, as previously defined, attached to the parent molecular moiety through an oxygen atom. Examples of alkoxy include, but are not limited to, methoxy, ethoxy, propoxy, isopropoxy, n-butoxy, sec-butoxy, tert-butoxy, n-pentoxy, neopentoxy, n-hexoxy and the like.

The term “substituted alkoxy,” as used herein, refers to an alkoxy group as previously defined substituted with one, two, three or more substituents.

The terms “halo” and “halogen,” as used herein, refer to an atom selected from fluorine, chlorine, bromine and iodine.

The terms “aryl” or “aromatic,” as used herein, refer to a mono- or polycyclic carbocyclic ring system having one or more aromatic rings. Examples of aryl groups include, but not limited to, phenyl, naphthyl, tetrahydronaphthyl, indanyl, idenyl and the like.

The terms “substituted aryl” or “substituted aromatic,” as used herein, refer to an aryl or aromatic group as previously defined substituted by one, two, three or more substituents.

The term “arylalkyl,” as used herein, refers to an aryl group attached to the parent molecular moiety via a C₁-C₃ alkyl or C₁-C₆ alkyl residue. Examples include, but are not limited to, benzyl, phenethyl and the like.

The term “substituted arylalkyl,” as used herein, refers to an arylalkyl group as previously defined, substituted by one, two, three or more substituents.

The terms “heteroaryl” or “heteroaromatic,” as used herein, refer to a mono-, bi-, or tri-cyclic aromatic radical or ring having from five to ten ring atoms of which at least one ring atom is selected from S, O and N; zero, one, two or three ring atoms are additional heteroatoms independently selected from S, O and N; and the remaining ring atoms are carbon, wherein any N or S contained within the ring may be optionally oxidized. Examples of heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like. The heteroaromatic ring may be bonded to the parent molecular moiety through a carbon or hetero atom.

The terms “substituted heteroaryl” or “substituted heteroaromatic,” as used herein, refer to a heteroaryl or heteroaromatic group as previously defined, substituted by one, two, three, or more substituents.

The term “alicyclic,” as used herein, denotes a monovalent group derived from a monocyclic or bicyclic saturated carbocyclic ring compound by the removal of a single hydrogen atom. Examples include, but are not limited to, cyclopropyl, cyclobutyl, cyclopentyl, cyclohexyl, bicyclo[2.2.1]heptyl, bicyclo[2.2.2]octyl and the like.

The term “substituted alicyclic,” as used herein, refers to an alicyclic group as previously defined, substituted by one, two, three or more substituents.

The terms “heterocyclic,” or “heterocycloalkyl” as used herein, refer to a non-aromatic ring, comprising three or more ring atoms, or a bi- or tri-cyclic fused system, where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, (iv) the nitrogen heteroatom may optionally be quaternized, (iv) any of the above rings may be fused to a benzene ring, and (v) the remaining ring atoms are carbon atoms which may be optionally oxo-substituted. Examples of heterocyclic groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, tetrahydrofuryl and the like.

The term “substituted heterocyclic,” as used herein, refers to a heterocyclic group, as previously defined, substituted by one, two, three or more substituents.

The term “heteroarylalkyl,” as used herein, refers to a heteroaryl group as previously defined, attached to the parent molecular moiety via an alkyl residue. Examples include, but are not limited to, pyridinylmethyl, pyrimidinylethyl and the like.

The term “substituted heteroarylalkyl,” as used herein, refers to a heteroarylalkyl group, as previously defined, substituted by one, two, three or more substituents.

The term “alkylamino,” as used herein, refers to a group having the structure —NH-alkyl.

The term “dialkylamino,” as used herein, refers to a group having the structure N(alkyl)₂ and cyclic amines. Examples of dialkylamino include, but are not limited to, dimethylamino, diethylamino, methylethylamino, piperidino, morpholino and the like.

The term “alkoxycarbonyl,” as used herein, refers to an ester group. i.e., an alkoxy group attached to the parent molecular moiety through a carbonyl group such as methoxycarbonyl, ethoxycarbonyl, and the like.

The term “carboxaldehyde,” as used herein, refers to a group of formula —CHO.

The term “carboxy,” as used herein, refers to a group of formula COOH.

The term “carboxamide,” as used herein, refers to a group of formula C(O)NH₂, C(O)N(H) alkyl or C(O)N (alkyl)₂, N(H)C(O) alkyl, N(alkyl)C(O) alkyl and the like.

The term “protecting group,” as used herein, refers to a labile chemical moiety which is known in the art to protect a hydroxyl, amino or thiol group against undesired reactions during synthetic procedures. After said synthetic procedure(s) the protecting group as described herein may be selectively removed. Protecting groups as known in the art are described generally in T. H. Greene and P. G. M. Wuts, Protective Groups in Organic Synthesis, 3rd edition, John Wiley & Sons, New York (1999). Examples of hydroxyl protecting groups include, but are not limited to, benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl (BOC), isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl (Alloc), acetyl (Ac), formyl, chloroacetyl, trifluoroacetyl, methoxyacetyl, phenoxyacetyl, benzoyl (Bz), methyl, t-butyl, 2,2,2-trichloroethyl, 2-trimethylsilyl ethyl, 1,1-dimethyl-2-propenyl, 3-methyl-3-butenyl, allyl, benzyl (Bn), para-methoxybenzyldiphenylmethyl, triphenylmethyl(trityl), 4,4′-dimethoxytriphenylmethyl (DMT), substituted or unsubstituted 9-(9-phenyl)xanthenyl(pixyl), tetrahydrofuryl, methoxymethyl, methylthiomethyl, benzyloxymethyl, 2,2,2-trichloroethoxymethyl, 2-(trimethylsilyl)ethoxymethyl, methanesulfonyl, para-toluenesulfonyl, trimethylsilyl, triethylsilyl, triisopropylsilyl, and the like. Preferred hydroxyl protecting groups for the present invention are DMT and substituted or unsubstituted pixyl.

Amino protecting groups as known in the art are described generally in T. H. Greene and P. G. M. Wuts, Protective Groups in Organic Synthesis, 3rd edition, John Wiley & Sons, New York (1999). Examples of amino protecting groups include, but are not limited to, t-butoxycarbonyl (BOC), 9-fluorenylmethoxycarbonyl (Fmoc), benzyloxycarbonyl, and the like.

Thiol protecting groups as known in the art are described generally in T. H. Greene and P. G. M. Wuts, Protective Groups in Organic Synthesis, 3rd edition, John Wiley & Sons, New York (1999). Examples of thiol protecting groups include, but are not limited to, triphenylmethyl (Trt), benzyl (Bn), and the like.

The term “protected hydroxyl group,” as used herein, refers to a hydroxyl group protected with a protecting group, as previously defined.

The term “protected amino group,” as used herein, refers to an amino group protected with a protecting group, as previously defined.

The term “protected thiol group,” as used herein, refers to a thiol group protected with a protecting group, as previously defined.

The term “acyl,” as used herein, refers to residues derived from substituted or unsubstituted acids including, but not limited to, carboxylic acids, carbamic acids, carbonic acids, sulfonic acids, and phosphorous acids. Examples include aliphatic carbonyls, aromatic carbonyls, aliphatic sulfonyls, aromatic sulfinyls, aliphatic sulfinyls, aromatic phosphates, aliphatic phosphates and the like.

The term “aprotic solvent,” as used herein, refers to a solvent that is relatively inert to proton activity, i.e., not acting as a proton-donor. Examples include, but are not limited to, hydrocarbons, such as hexane, toluene and the like, halogenated hydrocarbons, such as methylene chloride, ethylene chloride, chloroform, and the like, heterocyclic compounds, such as tetrahydrofuran, N-methylpyrrolidinone and the like, and ethers such as diethyl ether, bis-methoxymethyl ether and the like. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al, Vol. II, in the Techniques of Chemistry Series, John Wiley & Sons, NY, 1986. Aprotic solvents useful in the processes of the present invention include, but are not limited to, toluene, acetonitrile, DMF, THF, dioxane, MTBE, diethylether, NMP, acetone, hydrocarbons, and haloaliphatics.

The term “protic solvent” or “protogenic solvent,” as used herein, refers to a solvent that tends to provide protons, such as an alcohol, for example, methanol, ethanol, propanol, isopropanol, butanol, t-butanol, and the like. Those skilled in the art are familiar with such solvents, and will know that individual solvents or mixtures thereof may be preferred for specific compounds and reaction conditions, depending upon such factors as the solubility of reagents, reactivity of reagents and preferred temperature ranges, for example. Further discussions of protic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986

The term “leaving group,” as used herein, refers to any group that is the conjugate base of a strong acid. Leaving groups which are useful in the present invention include, but are not limited to, halogen, alkylsulfonyl, substituted alkylsulfonyl, arylsulfonyl, substituted arylsulfonyl, heterocyclcosulfonyl or trichloroacetimidate. A more preferred leaving groups of the present invention include chloro, fluoro, bromo, iodo, p-(2,4-dinitroanilino)benzenesulfonyl, benzenesulfonyl, methylsulfonyl(mesylate), p-methylbenzene-sulfonyl (tosylate), p-bromobenzenesulfonyl, trifluoromethyl-sulfonyl(triflate), trichloroacetimidate, acyloxy, 2,2,2-trifluoroethanesulfonyl, imidazolesulfonyl, and 2,4,6 trichlorophenyl, with chloro being preferred.

The term “Lewis acid,” as used herein, refers to, any species with a vacant electron orbital. Examples include, but are not limited to AlCl₃, BF₃, FeCl₃, SbF₅, SnCl₄, ZnCl₂, and ZnBr₂.

The synthesized compounds can be separated from a reaction mixture and further purified by a method such as column chromatography, high pressure liquid chromatography, precipitation, or recrystallization. Further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

Oligomeric Compounds

The term “oligonucleotide,” as used herein, refers to an oligomer or polymer of ribonucleic acid (RNA) or deoxyribonucleic acid (DNA) composed of naturally occurring nucleobases, sugars and phosphodiester internucleoside linkages.

As is known in the art, a nucleoside is a base-sugar combination. The base portion of the nucleoside is normally a heterocyclic base moiety. The two most common classes of such heterocyclic bases are purines and pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. Within the oligonucleotide structure, the phosphate groups are commonly referred to as forming the internucleoside linkages of the oligonucleotide, or in conjunction with the sugar ring, the backbone of the oligonucleotide. In forming oligonucleotides, the phosphate groups covalently link adjacent nucleosides to one another to form a linear polymeric compound. The normal internucleoside linkage of RNA and DNA is a 3′ to 5′ phosphodiester linkage.

The terms “oligomer” and “oligomeric compound,” as used herein, refer to a plurality of naturally-occurring or non-naturally-occurring nucleosides, joined together in a specific sequence, to form a polymeric structure capable of hybridizing a region of a nucleic acid molecule, (i.e. oligonucleotides that have one or more non-naturally occurring portions which function in a similar manner to oligonucleotides). The terms “oligomer” and “oligomeric compound” include oligonucleotides, oligonucleotide analogs, oligonucleotide mimetics, oligonucleosides and chimeric combinations of these, and are thus intended to be broader than the term “oligonucleotide,” including all oligomers having all manner of modifications known in the art. Oligomeric compounds are typically structurally distinguishable from, yet functionally interchangeable with, naturally-occurring or synthetic wild-type oligonucleotides. Thus, oligomeric compounds include all such structures that function effectively to mimic the structure and/or function of a desired RNA or DNA strand, for example, by hybridizing to a target. Such non-naturally occurring oligonucleotides are often desired over the naturally occurring forms because of desirable properties they can impart such as, for example, enhanced cellular uptake, enhanced affinity for nucleic acid target and increased stability in the presence of nucleases.

Thus, oligomeric compounds are typically prepared having enhanced properties compared to the native oligonucleotide analog, against nucleic acid targets. A target is identified and an oligonucleotide is selected having an effective length and sequence that is complementary to a portion of the target sequence. Each nucleoside of the selected sequence is scrutinized for possible enhancing modifications. A preferred modification would be the replacement of one or more RNA nucleosides with nucleosides that have the same 3′-endo conformational geometry. Such modifications can enhance chemical and nuclease stability relative to native RNA while at the same time being much cheaper and easier to synthesize and/or incorporate into an oligonulceotide. The selected sequence can be further divided into regions and the nucleosides of each region evaluated for enhancing modifications that can be the result of a chimeric configuration. Consideration is also given to the 5′- and 3′-termini as there are often advantageous modifications that can be made to one or more of the terminal nucleosides. Further modifications are also considered, such as internucleoside linkages, conjugate groups, substituted sugars or bases, replacing of one or more nucleosides with nucleoside mimetics and any other modification that can enhance the selected sequence for its intended target.

Oligomeric compounds are routinely prepared linearly but can be joined or otherwise prepared to be circular (by hybridization or by formation of a covalent bond) and may also include branching, however open linear structures are generally desired. Oligomeric compounds can include double stranded constructs such as for example two strands hybridized to form double stranded compounds. The double stranded compounds can be linked or separate and can include overhangs on the ends. In general, an oligomeric compound comprises a backbone of linked momeric subunits where each linked momeric subunit is directly or indirectly attached to a heterocyclic base moiety. Oligomeric compounds may also include monomeric subunits that are not linked to a heterocyclic base moiety thereby providing abasic sites. The linkages joining the monomeric subunits, the sugar moieties or surrogates and the heterocyclic base moieties can be independently modified giving rise to a plurality of motifs for the resulting oligomeric compounds including hemimers, gapmers and chimeras.

Further included in the present invention are oligomeric compounds such as antisense oligomeric compounds, antisense oligonucleotides, ribozymes, external guide sequence (EGS) oligonucleotides, alternate splicers, primers, probes, and other oligomeric compounds which hybridize to at least a portion of the target nucleic acid. As such, these oligomeric compounds may be introduced in the form of single-stranded, double-stranded, circular or hairpin oligomeric compounds and may contain structural elements such as internal or terminal bulges or loops. Once introduced to a system, the oligomeric compounds of the invention may elicit the action of one or more enzymes or structural proteins to effect modification of the target nucleic acid.

One non-limiting example of such an enzyme is RNAse H, a cellular endonuclease which cleaves the RNA strand of an RNA:DNA duplex. It is known in the art that single-stranded antisense oligomeric compounds which are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While one form of antisense oligomeric compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals and is believed to have an evolutionary connection to viral defense and transposon silencing.

A representative example of one type of oligomer synthesis that utilizes the coupling of an activated phosphorus group with a reactive hydroxyl group is the widely used phosphoramidite approach. A phosphoramidite monomeric subunit is reacted under appropriate conditions with a reactive hydroxyl group to form a phosphite linkage that is further oxidized to a phosphodiester or phosphorothioate linkage. This approach commonly utilizes nucleoside phosphoramidites of formula (IV): 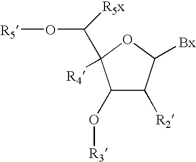
wherein R₅′ is DMT and R₃′ is —P(Pg)(Pn) and remainder of the variables are previously described.

Groups that are attached to the phosphorus atom of internucleotide linkages before and after oxidation (RN1) (RN2) can include nitrogen containing cyclic moieties such as morpholine. Such oxidized internucleoside linkages include a phosphoromorpholidothioate linkage (Wilk et al., Nucleosides and Nucleotides, 1991, 10, 319-322). Further cyclic moieties amenable to the present invention include mono-, bi- or tricyclic ring moieties which may be substituted with groups such as oxo, acyl, alkoxy, alkoxycarbonyl, alkyl, alkenyl, alkynyl, amino, amido, azido, aryl, heteroaryl, carboxylic acid, cyano, guanidino, halo, haloalkyl, haloalkoxy, hydrazino, ODMT, alkylsulfonyl, nitro, sulfide, sulfone, sulfonamide, thiol and thioalkoxy. A preferred bicyclic ring structure that includes nitrogen is phthalimido.

Some representative examples/combinations of Pn and Pg groups of formula (IV) which are known to one of ordinary skill in the art and are amenable to the present invention are shown below:

| Pn | Pg |
|---|---|
| 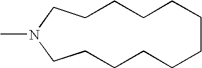 | —O—CH₃ |
| 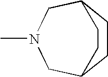 | —O—CH₃ |
| 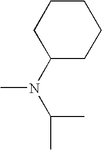 | —O—CH₃ |
| 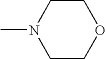 | 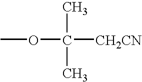 |
| 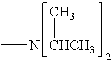 | —O—CH₂CH₂SiCH₃ |
| 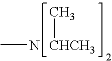 | 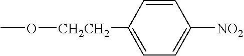 |
| —N(CH₃)₂ | 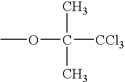 |
| —N(CH₂CH₃)₂ | 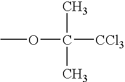 |
| 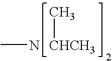 | 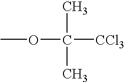 |
| 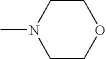 | 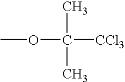 |
| —N(CH₃)₂ | —O—CH₂CCl₃ |
| 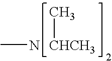 | —CH₂CH═CH₂ |
| 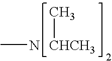 | —O—CH₂CH₂CN |

Further examples include:

| Pn | Pg |
|---|---|
| —N(CH₃)₂ | 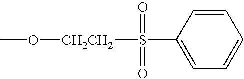 |
| 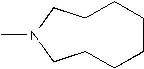 | 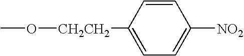 |
| 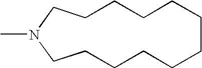 | 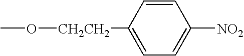 |
| 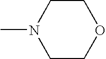 | 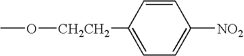 |
| 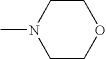 | 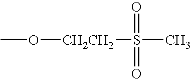 |
| 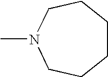 | 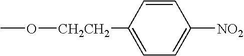 |
| 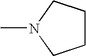 | —O—CH₃ |
| 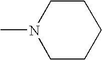 | —O—CH₃ |
| 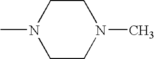 | —O—CH₃ |
| 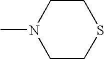 | —O—CH₃ |
| 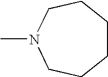 | —O—CH₃ |
| 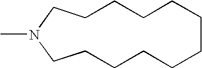 | —O—CH₃ |

Nucleobases and Modified Nucleobases

Oligomeric compounds may also include nucleobase (often referred to in the art simply as “base” or “heterocylic base moiety”). The terms “unmodified nucleobase” or “natural nucleobase,” as used herein refer oligomeric compounds containing one or more of the purine bases adenine (A) and guanine (G), and the pyrimidine bases thymine (T), cytosine (C) and uracil (U).

The term “modified nucleobase,” as used herein, refers to oligomeric compounds containing one or more other synthetic and natural nucleobases such as xanthine, hypoxanthine, 2-aminopyridine and 2-pyridone, 5-methylcytosine (5-me-C), 5-hydroxymethyl cytosine, 2-amino and 2-fluoroadenine, 2-propyl and other alkyl derivatives of adenine and guanine, 2-thio cytosine, uracil, thymine, 3-deaza guanine and adenine, 4-thiouracil, 5-uracil (pseudouracil), 5-propynyl (—C≡C—CH₃) uracil and cytosine and other alkynyl derivatives of pyrimidine bases, 5-halo particularly 5-bromo, 5-trifluoromethyl and other 5-substituted uracils and cytosines, 6-methyl and other alkyl derivatives of adenine and guanine, 6-azo uracil, cytosine and thymine, 7-methyl adenine and guanine, 7-deaza adenine and guanine, 8-halo, 8-amino, 8-aza, 8-thio, 8-thioalkyl, 8-hydroxyl and other 8-substituted adenines and guanines, universal bases, hydrophobic bases, promiscuous bases, size-expanded bases, and fluorinated bases as defined herein. Further modified nucleobases include tricyclic pyrimidines such as phenoxazine cytidine(1H-pyrimido[5,4-b][1,4]benzoxazin-2(3H)-one) and phenothiazine cytidine (1H-pyrimido[5,4-b][1,4]benzothiazin-2(3H)-one).

Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993.

Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyl-adenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,681,941, and 5,750,692.

The term “universal base” as used herein, refers to a moiety that may be substituted for any base. The universal base need not contribute to hybridization, but should not significantly detract from hybridization and typically refers to a monomer in a first sequence that can pair with a naturally occurring base, i.e A, C, G, T or U at a corresponding position in a second sequence of a duplex in which one or more of the following is true: (1) there is essentially no pairing between the two; or (2) the pairing between them occurs non-discriminantly with each of the naturally occurring bases and without significant destabilization of the duplex. Exemplary universal bases include, without limitation, inosine, 5-nitroindole and 4-nitrobenzimidazole.

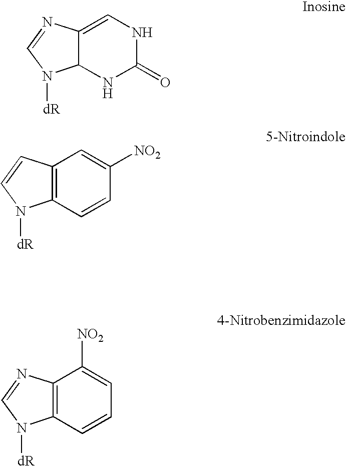

Additional examples of universal bases include, but are not limited to, those shown below. For further examples and descriptions of universal bases see Survey and summary: the applications of universal DNA base analogs. Loakes, D. Nucleic Acids Research, 2001, 29, 12, 2437-2447.

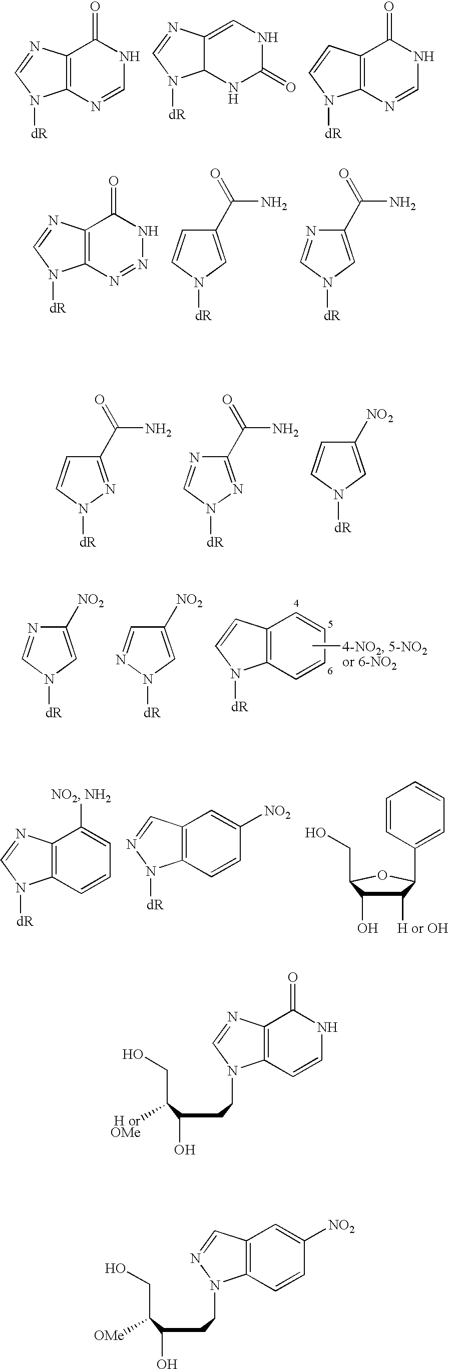

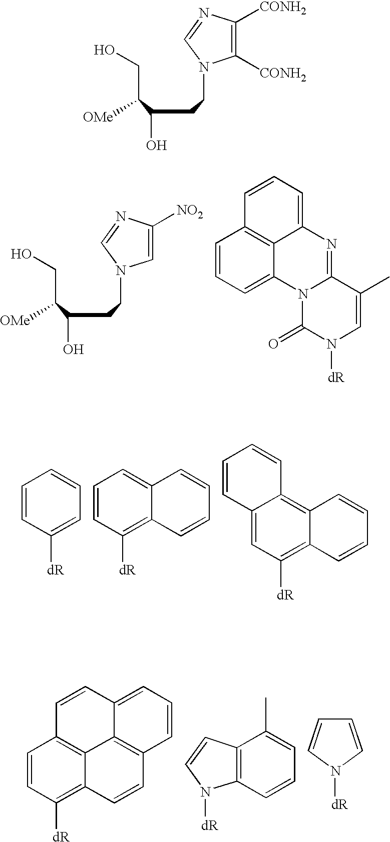

The term “hydrophobic base” as used herein, refers to a monomer in a first sequence that can pair with a naturally occurring base, i.e A, C, G, T or U at a corresponding position in a second sequence of a duplex in which one or more of the following is true: (1) the hydrophobic base acts as a non-polar close size and shape mimic (isostere) of one of the naturally occurring nucleosides; or (2) the hydrophobic base lacks all hydrogen bonding functionality on the Watson-Crick pairing edge.

Examples of adenine isosteres include, but are not limited to those shown below. For further examples and definitions of adenine isosteres see Probing the requirements for recognition and catalysis in Fpg and MutY with nonpolar adenine isosteres. Francis, A W, Helquist, S A, Kool, E T, David, S S. J. Am. Chem. Soc., 2003, 125, 16235-16242 or Structure and base pairing properties of a replicable nonpolar isostere for deoxyadenosine. Guckian, K M, Morales, J C, Kool, E T. J. Org. Chem., 1998, 63, 9652-96565.

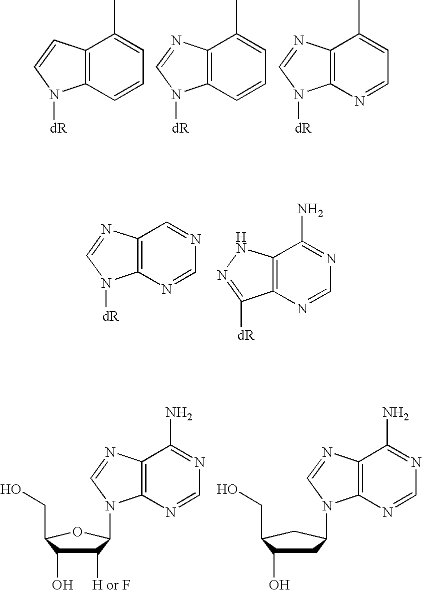

A non-limiting example of a cytosine isostere is 2-fluoro-4-methylbenzene deoxyribonucleoside, shown below. For additional information on cytosine isosteres see Hydrolysis of RNA/DNA hybrids containing nonpolar pyrimidine isosteres defines regions essential for HIV type polypurine tract selection. Rausch, J W, Qu, J, Yi-Brunozzi H Y, Kool, E T, LeGrice, S F J. Proc. Natl. Acad. Sci., 2003, 100, 11279-11284.

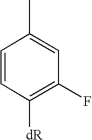

A non-limiting example of a guanosine isostere is 4-fluoro-6-methylbenzimidazole deoxyribonucleoside, shown below. For additional information on guanosine isosteres, see A highly effective nonpolar isostere of doeoxguanosine: synthesis, structure, stacking and base pairing. O'Neil, B M, Ratto, J E, Good, K L, Tahmassebi, D C, Helquist, S A, Morales, J C, Kool, E T. J. Org. Chem., 2002, 67, 5869-5875.

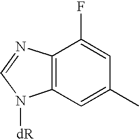

A non-limiting example of a thymidine isostere is 2,4-difluoro-5-toluene deoxyribonucleoside, shown below. For additional information on thymidine isosteres see A thymidine triphosphate shape analog lacking Watson-Crick pairing ability is replicated with high sequence selectivity. Moran, S, Ren, R X-F, Kool, E T. Proc. Natl. Acad. Sci., 1997, 94, 10506-10511 or Difluorotoluene, a nonpolar isostere for thymidine, codes specifically and efficiently for adenine in DNA replication. J. Am. Chem. Soc. 1997, 119, 2056-2057.

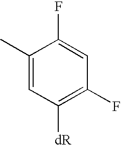

The term “promiscuous base” as used herein, refers to a monomer in a first sequence that can pair with a naturally occurring base, i.e A, C, G, T or U at a corresponding position in a second sequence of a duplex in which the promiscuous base can pair non-discriminantly with more than one of the naturally occurring bases, i.e. A, C, G, T, U. Non-limiting examples of promiscuous bases are 6H,8H-3,4-dihydropyrimido[4,5-c][1,2]oxazin-7-one and N⁶-methoxy-2,6-diaminopurine, shown below. For further information, see Polymerase recognition of synthetic oligodeoxyribonucleotides incorporating degenerate pyrimidine and purine bases. Hill, F.; Loakes, D.; Brown, D. M. Proc. Natl. Acad. Sci., 1998, 95, 4258-4263.

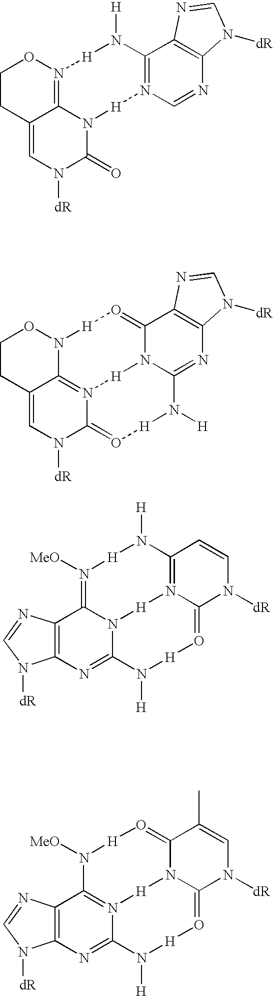

The term “size expanded base” as used herein, refers to analogs of naturally occurring nucleobases that are larger in size and retain their Watson-Crick pairing ability. Tow non-limiting examples of size-expanded bases are shown below. For further discussions of size expanded bases see A four-base paired genetic helix with expanded size. Liu, B, Gao, J, Lynch, S R, Saito, D, Maynard, L, Kool, E T., Science, 2003, 302, 868-871 and Toward a new genetic system with expanded dimension; size expanded analogues of deoxyadenosine and thymidine. Liu, H, Goa, J, Maynard, Y, Saito, D, Kool, E T, J. Am. Chem. Soc. 2004, 126, 1102-1109 and Expanded-Size Bases in Naturally Sized DNA: Evaluation of Steric Effects in Watson-Crick Pairing. Gao, J, Liu, H, Kool, E, J. Am. Chem. Soc. 2004, 126, 11826-11831.

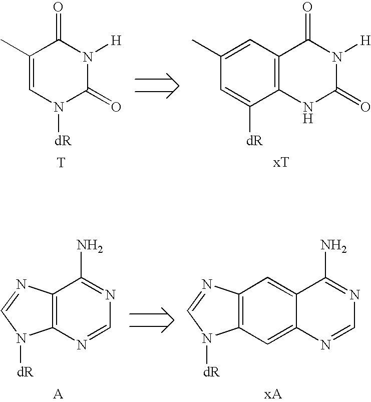

The term “fluorinated nucleobase” as used herein, refers to a nucleobase or nucleobase analog, wherein one or more of the aromatic ring substituents is a fluoroine atom. It may be possible that all of the ring substituents are fluoroine atoms. Some non-limiting examples of fluorinated nucleobase are shown below. For further examples of fluorinated nucleobases see fluorinated DNA bases as probes of electrostatic effects in DNA base stacking. Lai, J S, QU, J, Kool, E T, Angew. Chem. Int. Ed., 2003, 42, 5973-5977 and Selective pairing of polyfluorinated DNA bases, Lai, J S, Kool, E T, J. Am. Chem. Soc., 2004, 126, 3040-3041 and The effect of universal fluorinated nucleobases on the catalytic activity of ribozymes, Kloppfer, A E, Engels, J W, Nucleosides, Nucleotides & Nucleic Acids, 2003, 22, 1347-1350 and Synthesis of 2′aminoalkyl-substituted fluorinated nucleobases and their influence on the kinetic properties of hammerhead ribozymes, Klopffer, A E, Engels, J W, Chem Bio Chem., 2003, 5, 707-716.

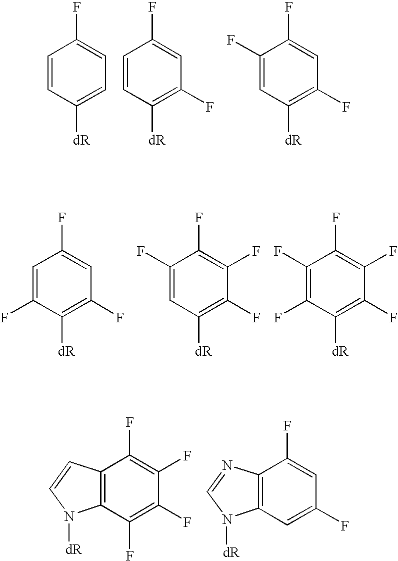

Other modified nucleobases include polycyclic heterocyclic moieties, which are routinely used in antisense applications to increase the binding properties of the modified strand to a target strand. The most studied modifications are targeted to guanosines hence they have been termed G-clamps or cytidine analogs.

Examples of G-clamps include substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one) and pyridoindole cytidine (H-pyrido[3′,2′:4,5]pyrrolo[2,3-d]pyrimidin-2-one).

Representative cytosine analogs that make 3 hydrogen bonds with a guanosine in a second oligonucleotide include 1,3-diazaphenoxazine-2-one (Kurchavov, et al., Nucleosides and Nucleotides, 1997, 16, 1837-1846), 1,3-diazaphenothiazine-2-one (Lin, K.-Y.; Jones, R. J.; Matteucci, M. J. Am. Chem. Soc. 1995, 117, 3873-3874) and 6,7,8,9-tetrafluoro-1,3-diazaphenoxazine-2-one (Wang, J.; Lin, K.-Y., Matteucci, M. Tetrahedron Lett. 1998, 39, 8385-8388). When incorporated into oligonucleotides these base modifications hybridized with complementary guanine (the latter also hybridized with adenine) and enhanced helical thermal stability by extended stacking interactions (see U.S. patent application Ser. No. 10/013,295).

Further helix-stabilizing properties have been observed for cytosine analogs comprising an aminoethoxy moiety attached to a rigid 1,3-diazaphenoxazine-2-one scaffold (Lin, K.-Y.; Matteucci, M. J. Am. Chem. Soc. 1998, 120, 8531-8532). A single incorporation can enhance the binding affinity of a model oligonucleotide to its complementary target DNA or RNA with an increase in ΔTₘ of up to 180 relative to 5-methyl cytosine (dC5^{me}), which is the highest known affinity enhancement for a single modification, yet. Conveniently, the gain in helical stability does not compromise the specificity of the oligonucleotides.

Further tricyclic, tetracyclic heteroaryl and polycyclic nucleobase analogs that are amenable to the present invention are disclosed in U.S. Pat. Nos. 5,434,257; 5,502,177; 5,646,269; 6,028,183, and 6,007,992, and U.S. patent application Ser. No. 09/996,292.

The enhanced binding affinity of these derivatives together with their uncompromised sequence specificity makes them valuable nucleobase analogs for the development of more potent antisense-based drugs. In vitro experiments demonstrated that heptanucleotides containing phenoxazine substitutions are able to activate RNaseH, enhance cellular uptake and increase antisense activity (Lin, K.-Y.; Matteucci, M. J. Am. Chem. Soc. 1998, 120, 8531-8532). The activity enhancement was even more pronounced for a single G-clamp substitution, which significantly improved the in vitro potency of a 20-mer 2′-deoxyphosphorothioate oligonucleotide (Flanagan, W. M.; Wolf, J. J.; Olson, P.; Grant, D.; Lin, K.-Y.; Wagner, R. W.; Matteucci, M. Proc. Natl. Acad. Sci. USA, 1999, 96, 3513-3518). Nevertheless, to optimize oligonucleotide design and better understand the impact of these heterocyclic modifications on biological activity, it is important to evaluate their effect on the nuclease stability of the oligomers.

Modified Sugars

The term “modified sugar,” as used herein, refers to oligomeric compounds containing one or more furanose rings that have been in some way altered. The heterocyclic base moiety or modified heterocyclic base moiety is maintained for hybridization with an appropriate target nucleic acid. Such “modified sugars” are often desired over the naturally occurring forms because of advantageous properties they can impart such as, for example, enhanced cellular uptake, enhanced affinity for nucleic acid target and increased stability in the presence of nucleases. The modifications to the furanose ring typically fall into two categories; those where the ring itself is altered, and those where the 5 membered furanose ring remains intact but is further substituted with novel groups.

The terms used to describe the conformational geometry of homoduplex nucleic acids are “A Form” for RNA and “B Form” for DNA, (determined from X-ray diffraction analysis of nucleic acid fibers, see Arnott et al., Biochem. Biophys. Res. Comm., 1970, 47, 1504). In general, RNA:RNA duplexes are more stable and have higher melting temperatures (Tm's) than DNA:DNA duplexes (Sanger, Principles of Nucleic Acid Structure, 1984, Springer-Verlag; New York, N.Y.; Lesnik et al., Biochemistry, 1995, 34, 10807-10815; Conte et al., Nucleic Acids Res., 1997, 25, 2627-2634). The increased stability of RNA has been attributed to several structural features, most notably the improved base stacking interactions that result from an A-form geometry (Searle et al., Nucleic Acids Res., 1993, 21, 2051-2056). The presence of the 2′ hydroxyl in RNA biases the sugar toward a C3′ endo pucker (also designated a Northern pucker), which causes the duplex to favor the A-form geometry. The 2′ hydroxyl groups of RNA also form a network of water mediated hydrogen bonds that help stabilize the RNA duplex (Egli et al., Biochemistry, 1996, 35, 8489-8494). Deoxy nucleic acids prefer a C2′ endo sugar pucker, (Southern pucker) imparting a less stable B-form geometry (Sanger, Principles of Nucleic Acid Structure, 1984, Springer-Verlag; New York).

DNA:RNA hybrid duplexes are usually less stable than pure RNA:RNA duplexes, and depending on their sequence, may be either more or less stable than DNA:DNA duplexes (Searle et al., Nucleic Acids Res., 1993, 21, 2051-2056). The structure of a hybrid duplex is intermediate between A- and B-form geometries, which may result in poor stacking interactions (Lane et al., Eur. J. Biochem., 1993, 215, 297-306; Fedoroff et al., J. Mol. Biol., 1993, 233, 509-523; Gonzalez et al., Biochemistry, 1995, 34, 4969-4982; Horton et al., J. Mol. Biol., 1996, 264, 521-533).

The stability of the duplex formed between a target RNA strand and a synthetic oligomeric strand is central to therapies such as, but not limited, to antisense and RNA interference. In the case of antisense, effective mRNA inhibition requires a very high binding affinity between the strands, while the triggering of RNA interference requires A form duplex geometry (i.e. predominantly 3′-endo). Other properties that are enhanced by using more stable 3′-endo nucleosides include but aren't limited to modulation of pharmacokinetic properties through modification of protein binding, protein off-rate, absorption and clearance; modulation of nuclease stability as well as chemical stability; modulation of the binding affinity and specificity of the oligomer (affinity and specificity for enzymes as well as for complementary sequences); and increasing efficacy of RNA cleavage. Hence a 3′-endo sugar orientation is highly desirable.

The preferred conformation of modified nucleosides and their oligomers can be estimated by various methods such as molecular dynamics calculations, nuclear magnetic resonance spectroscopy and CD measurements. Hence, modifications predicted to induce a 3′-endo sugar conformation (i.e. A-form duplex geometry in an oligomeric context), are selected for use in the modified oligonucleotides of the present invention. A nucleoside can incorporate synthetic modifications of the heterocyclic base, the sugar moiety or both to induce a desired 3′-endo sugar conformation The syntheses of numerous of the modified nucleosides amenable to the present invention are known in the art (see for example, Chemistry of Nucleosides and Nucleotides Vol 1-3, ed. Leroy B. Townsend, 1988, Plenum press).

Nucleoside conformation is influenced by substitution at the 2′, 3′ or 4′-positions of the pentofuranosyl sugar. Electronegative substituents generally prefer the axial positions, while sterically demanding substituents generally prefer the equatorial positions (Sanger, Principles of Nucleic Acid Structure, 1984, Springer-Verlag; New York.) Modification of the 2′ position to favor the 3′-endo conformation can be achieved while maintaining the 2′-OH as a recognition element, (Gallo et al., Tetrahedron, 2001, 57, 5707-5713; Harry-O'kuru et al., J. Org. Chem., 1997, 62(6), 1754-1759 and Tang et al., J. Org. Chem., 1999, 64, 747-754.) Alternatively, preference for the 3′-endo conformation can be achieved by deletion of the 2′-OH as exemplified by 2′deoxy-2′-F-nucleosides (Kawasaki et al., J. Med. Chem., 1993, 36, 831-841), which adopt the 3′-endo conformation placing the electronegative fluorine atom in the axial position. Other substitutions of the ribose ring, for example substitution at the 4′-position to give 4′-F modified nucleosides (Guillerm et al., Bioorg and Med. Chem. Lett., 1995, 5, 1455-1460 and Owen et al., J. Org. Chem., 1976, 41, 3010-3017), also induce preference for the 3′-endo conformation.

Substitution of the sugar at the 2′-position with a substituent group that influences the sugar geometry, is a routinely used method of modifying the sugar puckering. The influence on ring conformation is dependant on the nature of the substituent at the 2′-position. A number of different substituents have been studied to determine their sugar puckering effect. For example, 2′-halogens have been studied showing that the 2′-fluoro derivative exhibits the largest population (65%) of the C3′-endo form, and the 2′-iodo exhibits the lowest population (7%). The populations of adenosine (2′-OH) versus deoxyadenosine (2′-H) are 36% and 19%, respectively. Additionally, the effect of the 2′-fluoro group on adenosine dimers (2′-deoxy-2′-fluoroadenosine-2′-deoxy-2′-fluoroadenosine) is further correlated to the stabilization of the stacked conformation.

Thus, relative duplex stability can be enhanced by replacement of 2′-OH groups with 2′-F groups thereby increasing the C3′-endo population. It is assumed that the highly polar nature of the 2′-F bond and the extreme preference for C3′-endo puckering may stabilize the stacked conformation in an A-form duplex. Data from UV hypochromicity, circular dichroism, and ¹H NMR also indicate that the degree of stacking decreases as the electronegativity of the halo substituent decreases. Furthermore, steric bulk at the 2′-position of the sugar moiety is better accommodated in an A-form duplex than a B-form duplex. Thus, a 2′-substituent on the 3′-terminus of a dinucleoside monophosphate is thought to exert a number of effects on the stacking conformation: steric repulsion, furanose puckering preference, electrostatic repulsion, hydrophobic attraction, and hydrogen bonding capabilities. These substituent effects are thought to be determined by the molecular size, electronegativity, and hydrophobicity of the substituent.

The term “substituted sugar” or “substituted sugar moiety,” as used herein, refers to the sugar moiety of an oligomeric compound that contains additional substituents. Oligomeric compounds of the invention may contain one or more substituted sugar moieties. These substituted sugar moieties can contain one, two, three, four or five substituents, at any position(s) on the sugar ring (namely 1′-, 2′-, 3′-, or 4′-). Preferred substitutions may be made at the 5′-position of the 5′ terminal nucleotide, the 3′-position of the 3′ terminal nucleoside or the 3′-position of a 2′-5′ linked oligonucleotide. Most preferably the substitution is in the 2′-position. 2′-sugar substituent groups may be in the arabino (up) position or ribo (down) position.

It is understood that naturally occurring deoxynucleotides contain no substituent at the 2′-position (i.e. they have two hydrogen atoms), while nucleotides derived from RNA will have one hydroxy group and one hydrogen atom at the 2′-position. Hence a 2′-H substituent refers to a DNA derivative and a 2′-OH would refer to an RNA derivative. It should be noted that a 2′-substituent can also be referred to as a 2′-deoxy-2′-substituent.

Suitable sugar substituents include, but are not limited to: OH, F, Cl, Br, SH, CN, OCN, CF₃, OCF₃, SOCH₃, SO₂CH₃, ONO₂, NO₂, N₃, NH₂, O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl, substituted or unsubstituted C₁ to C₁₀ alkyl, substituted or unsubstituted C₂ to C₁₀ alkenyl, substituted or unsubstituted alkynyl, alkaryl, aralkyl, O-alkaryl or O-aralkyl, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties.

Preferred sugar substituents are selected from: OH, F, O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl, including O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙH]₂, where n and m are from 1 to about 10.

Other preferred substituents in the 2′-position include 2′-fluoro, 2′-methoxy, 2′-aminopropoxy (2′-OCH₂CH₂CH₂NH₂), 2′-allyl (2′-CH₂—CH═CH₂), 2′-O-allyl (2′-O—CH₂—CH═CH₂), 2′-methoxyethoxy(2′-OCH₂CH₂OCH₃, also known as 2′-O-(2-methoxyethyl) or 2′-MOE) (Martin et al., Helv. Chim. Acta, 1995, 78, 486-504), 2′-dimethylaminooxyethoxy(2′-O(CH₂)₂ON(CH₃)₂ or 2′-DMAOE), and 2′-dimethylaminoethoxyethoxy(2′-O—(CH₂)₂—O—(CH₂)₂—N(CH₃)₂, also known as 2′-O-dimethyl-amino-ethoxyethyl or 2′-DMAEOE).

Oligonucleotides having the 2′-MOE side chain (Baker et al., J. Biol. Chem., 1997, 272, 11944-12000) demonstrate a very high binding affinity (greater than many similar 2′ modifications such as O-methyl, O-propyl, and O-aminopropyl), increased nuclease resistance, and have shown antisense inhibition of gene expression with promising features for in vivo use (Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides and Nucleotides, 1997, 16, 917-926). Chimeric oligonucleotides having 2′-MOE substituents in the wing nucleosides and an internal region of deoxy-phosphorothioate nucleotides (also termed a gapped oligonucleotide or gapmer) have shown effective reduction in the growth of tumors in animal models at low doses. 2′-MOE substituted oligonucleotides have also shown outstanding promise as antisense agents in several disease states. One such MOE substituted oligonucleotide is presently being investigated in clinical trials for the treatment of CMV retinitis.

Further representative sugar substituents include groups of formula Ia or Ib:

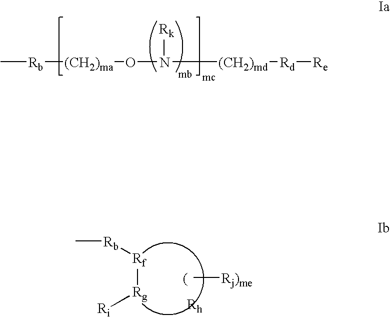

wherein:

R_{b} is O, S or NH;

R_{d} is a single bond, O, S or C(═O);

Rₑ is C₁-C₁₀ alkyl, N(Rₖ)(Rₘ), N(Rₖ)(Rₙ), N═C(Rₚ)(R_{q}), N═C(Rₚ)(Rᵣ) or has formula Ic;

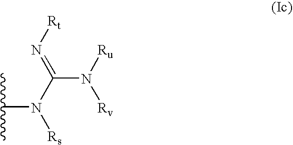

Rₚ and R_{q} are each independently hydrogen or C₁-C₁₀ alkyl;

Rᵣ is —Rₓ—R_{y};

each Rₛ, Rₜ, Rᵤ, and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;

or optionally, Rᵤ and Rᵥ, together form a phthalimido moiety with the nitrogen atom to which they are attached;

each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;

Rₖ is hydrogen, an amino protecting group or —Rₓ—R_{y};

Rₚ is hydrogen, an amino protecting group or —Rₓ—R_{y};

Rₓ is a bond or a linking moiety;

R_{y} is a chemical functional group, a conjugate group or a solid support medium;

each Rₘ and Rₙ is, independently, H, an amino protecting group, substituted or unsubstituted C₁-C₁₀ alkyl, substituted or unsubstituted C₂-C₁₀ alkenyl, substituted or unsubstituted C₂-C₁₀ alkynyl,

wherein the substituent groups are selected from hydroxyl, amino, alkoxy, carboxy, benzyl, phenyl, nitro, thiol, thioalkoxy, halogen, alkyl, aryl, alkenyl, alkynyl; NH₃⁺, N(Rᵤ)(Rᵥ), guanidino and acyl where said acyl is an acid amide or an ester;

or Rₘ and Rₙ, together, are an amino protecting group, are joined in a ring structure that optionally includes an additional heteroatom selected from N and O or are a chemical functional group;

Rᵢ is OR_{z}, SR_{z}, or N(R_{z})₂;

each R_{z} is, independently, H, C₁-C₈ alkyl, C₁-C₈ haloalkyl, C(═NH)N(H)Rᵤ, C(═O)N(H)Rᵤ or OC(═O)N(H)Rᵤ;

R_{f}, R_{g} and Rₕ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;

Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ)ORₖ, halo, SRₖ or CN;

ma is 1 to about 10;

each mb is, independently, 0 or 1;

mc is 0 or an integer from 1 to 10;

md is an integer from 1 to 10;

me is from 0, 1 or 2; and

provided that when mc is 0, md is greater than 1.

Representative substituent groups of Formula Ia, Formula Ib, Formula Ic are disclosed in U.S. patent application Ser. Nos. 09/130,973, 09/123,108, and 09/349,040 respectively. Representative acetamido substituent groups are disclosed in U.S. Pat. No. 6,147,200, and dimethylaminoethyloxyethyl substituent groups are disclosed in International Patent Application PCT/US99/17895.

Some representative examples of substituted nucleosides amenable to the present invention include, but are not limited to those shown below:

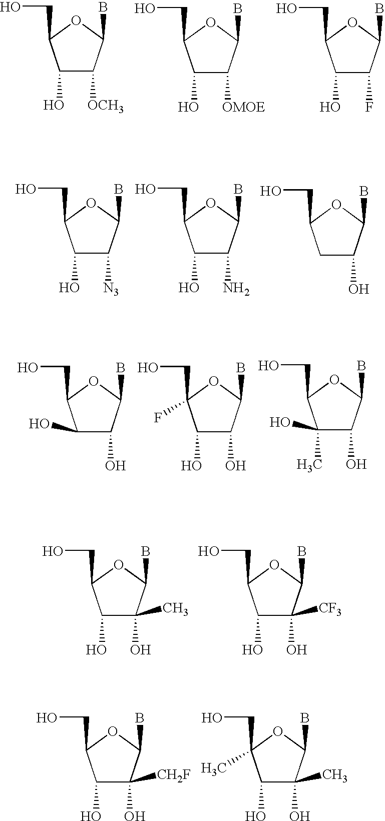

Sugars having 4′-O-substitutions on the ribosyl ring are also amenable to the present invention. Representative substitutions for ring 0 include S, CH₂, CHF, and CF₂, see, e.g., Secrist, et al., Abstract 21, Program & Abstracts, Tenth International Roundtable, Nucleosides, Nucleotides and their Biological Applications, Park City, Utah, Sep. 16-20, 1992, hereby incorporated by reference in its entirety.

The terms “sugar mimetic” and “sugar surrogate,” as used herein, refer to oligomeric compounds wherein the furanose ring is replaced with a novel group, which is often desired over the naturally occurring forms because of advantageous properties they can impart, as previously described. One of skill in the art can envisage many ways to replace the furanose ring. Some examples include, but are not limited to those given below.

Bicylco[3.1.0]hexane (methanocarba) nucleoside analogs, in which the furanose ring is replaced with a cylcopropane/cyclopentane bicyclic moiety can induce the 2′-exo or 3′-exo conformation, depending on structure, (Maier et al., Nucleic Acids Research. 2004, 32(12), 3642-3650). A 16-mer oligonucleotide, incorporating ten bicyclo[3.1.0]hexane pseudosugar rings fixed in a Northern conformation, resulted in an increase in Tm (Marquez et al., J. Med. Chem. 1996, 39, 3719-3747).

Oligonucleotide mimetics have been prepared to include bicyclic and tricyclic sugar analogs (Steffens et al., Helv. Chim. Acta, 1997, 80, 2426-2439; Steffens et al., J. Am. Chem. Soc., 1999, 121, 3249-3255; and Renneberg et al., J. Am. Chem. Soc., 2002, 124, 5993-6002). The tricyclic analogs showed increased thermal stabilities (Tm's) when hybridized to DNA, RNA and itself, while the bicyclic analogs showed thermal stabilities approaching that of DNA duplexes.

Another oligonucleotide mimetic has been reported wherein the furanosyl ring has been replaced by a cyclobutyl moiety (see U.S. Pat. No. 3,539,044).

Representative U.S. patents that teach the preparation of such modified sugar structures include, but are not limited to, U.S. Pat. Nos. 4,981,957; 5,118,800; 5,319,080; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920.

Preferred nucleosides having bicyclic sugar moieties include “Locked Nucleic Acids” (LNAs) in which the 2′-hydroxyl group of the ribosyl sugar ring is linked to the 4′ carbon atom, thereby forming a 2′-C,4′-C-oxymethylene linkage to form a bicyclic sugar moiety (reviewed in Elayadi et al., Curr. Opinion Invens. Drugs, 2001, 2, 558-561; Braasch et al., Chem. Biol., 2001, 8, 1-7; and Orum et al., Curr. Opinion Mol. Ther., 2001, 3, 239-243; see also U.S. Pat. Nos. 6,268,490 and 6,670,461). The term locked nucleic acid has also been used in a broader sense in the literature to include any bicyclic structure that locks the sugar conformation. LNA's are commercially available from ProLigo (Paris, France and Boulder, Colo., USA).

2D NMR spectroscopy revealed that the locked orientation of the LNA nucleotides (single-stranded and duplex), constrains the phosphate backbone to a higher population of the 3′-endo conformation (Petersen et al., J. Mol. Recognit., 2000, 13, 44-53, and Wengel et al., Nucleosides and Nucleotides, 1999, 18, 1365-1370). LNA:LNA hybridization forms exceedingly stable duplexes, which have been shown to be the most thermally stable nucleic acid type duplex system (Koshkin et al., J. Am. Chem. Soc., 1998, 120, 13252-13253). LNA analogs also display very high duplex thermal stabilities with complementary DNA and RNA (Tm=+3 to +10 C), stability towards 3′-exonucleolytic degradation and good solubility properties. Antisense oligonucleotides containing LNAs can confer several desired properties to antisense agents (Wahlestedt et al., Proc. Natl. Acad. Sci. U.S.A., 2000, 97, 5633-5638). LNA:DNA copolymers were not degraded readily in blood serum and cell extracts, and exhibited potent antisense activity in assay systems as disparate as G-protein-coupled receptor signaling in living rat brain and detection of reporter genes in Escherichia coli. Lipofectin-mediated efficient delivery of LNA into living human breast cancer cells has also been accomplished. DNA:LNA chimeras have been shown to efficiently inhibit gene expression when targeted to a variety of regions (e.g. 5′-untranslated, start codon or coding regions) within the luciferase mRNA (Braasch et al., Nucleic Acids Research, 2002, 30, 5160-5167). Further successful in vivo studies involving LNA's have shown knock-down of the rat delta opioid receptor without toxicity (Wahlestedt et al., Proc. Natl. Acad. Sci., 2000, 97, 5633-5638) and blockage of the translation of the large subunit of RNA polymerase II (Fluiter et al., Nucleic Acids Res., 2003, 31, 953-962).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630 and WO 98/39352 and WO 99/14226).

Phosphorothioate-LNA, 2′-thio-LNA (Kumar et al., Bioorg. Med. Chem. Lett., 1998, 8, 2219-2222), and 2′-amino-LNA (Singh et al., J Org. Chem., 1998, 63, 10035-10039) have also been prepared.

An isomer of LNA, is □-L-LNA which shows superior stability against a 3′-exonuclease (Frieden et al., Nucleic Acids Research, 2003, 21, 6365-6372), and when incorporated into antisense gapmers and chimeras showed potent antisense activity.

Preferred nucleosides having bicyclic sugar moieties also include ENA™ where an extra methylene group is added to the bridge to give 2′-O,4′-ethylene-bridged nucleic acid ENA™, (Singh et al., Chem. Commun., 1998, 4, 455-456 and Morita et al., Bioorganic Medicinal Chemistry, 2003, 11, 2211-2226). ENA™'s have similar properties to LNA's showing enhanced affinity for DNA/RNA, high resistance to nuclease degradation and have been studied as antisense nucleic acids (see: Morita et al., Bioorg Med. Chem., 2002, 12, 73-76; Morita et al., Bioorg Med. Chem., 2003, 11, 2211-2226; Morita et al., Nucleic Acids Res. Suppl., 2002, Suppl. 2, 99-100; Morita et al., Nucleosides, Nucleotides & Nucleic Acids., 2003, 22, 1619-1621; and Takagi et al., Nucleic Acids Res. Supp., 2003, 3, 83-84). ENA™'s are commercially available from Sigma Genosys Japan.

A similar bicyclic sugar moiety that has been prepared and studied has the bridge going from the 3′-hydroxyl group via a single methylene group to the 4′ carbon atom of the sugar ring thereby forming a 3′-C,4′-C-oxymethylene linkage (3′,4′-BNA; see U.S. Pat. No. 6,043,060). The nitrogen containing analog (3′-amino-3′,4′-BNA) has also been prepared and shown to adopt a Southern type conformation (see Obika et al., Tetrahedron Lett., 2003, 44, 5267-5270). Another bicyclic sugar analog has the bridge going from the 2′-hydroxyl group via a single methylene group to the 1′ carbon atom of the sugar ring thereby forming a 2′-C,1′-C-oxymethylene linkage (1′,2′-oxetane; see Pushpangadan et al., J. Am. Chem. Soc., 2004, 126, 11484-11499)

These furanosyl sugar mimetics can be considered as repeating units of the general structure shown below:

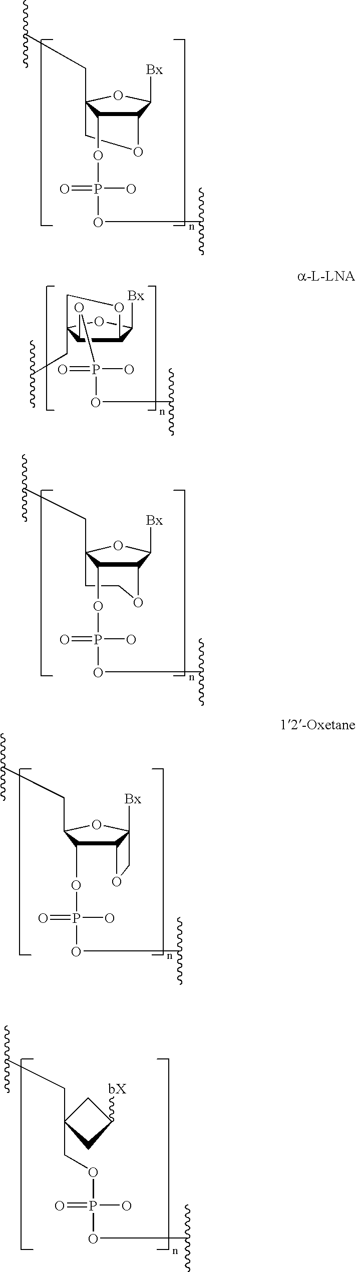

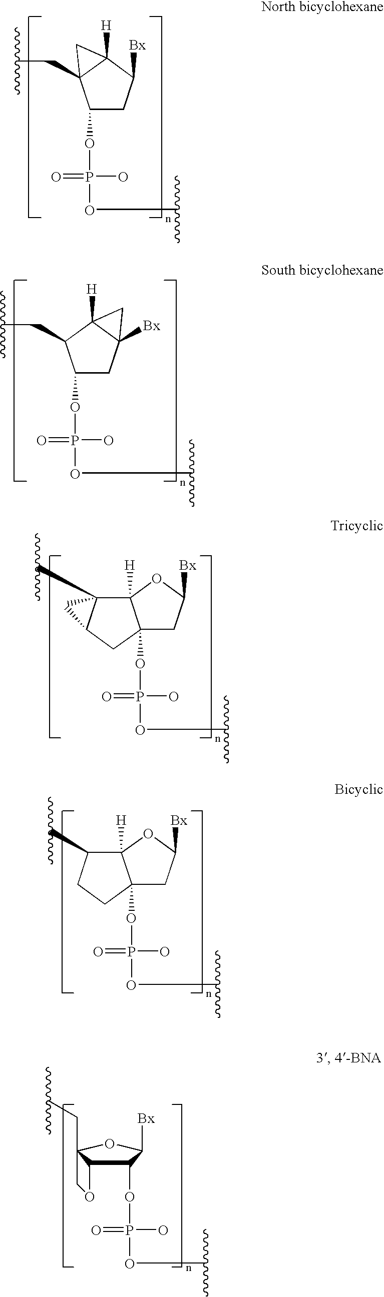

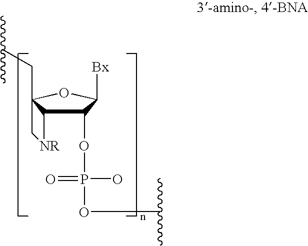

wherein

each Bx is independently a nucleobase,

n is from 1 to about 40 and

[symbol]

represents connection to the next monormeric unit, or end terminus.

Modified Internucleoside Linkages

The terms “modified internucleoside linkage” or “modified oligonucleotide backbone,” as used herein, refers to oligonucleotides containing non-naturally occurring internucleoside linkages (i.e. non-phosphodiester linkages), including internucleoside linkages that retain a phosphorus atom and internucleoside linkages that do not have a phosphorus atom.

The term “oligonucleoside,” as used herein, refers to a sequence of nucleosides that are joined by internucleoside linkages that do not have phosphorus atoms. Internucleoside linkages of this type include short chain alkyl, cycloalkyl, mixed heteroatom alkyl, mixed heteroatom cycloalkyl, one or more short chain heteroatomic and one or more short chain heterocyclic. These internucleoside linkages include but are not limited to siloxane, sulfide, sulfoxide, sulfone, acetyl, formacetyl, thioformacetyl, methylene formacetyl, thioformacetyl, alkeneyl, sulfamate; methyleneimino, methylenehydrazino, sulfonate, sulfonamide, amide and others having mixed N, O, S and CH₂ component parts. Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439.

In the C. elegans system, modification of the internucleotide linkage (phosphorothioate in place of phosphodiester) did not significantly interfere with RNAi activity, indicating that oligomeric compounds of the invention can have one or more modified internucleoside linkages, and retain activity. Indeed, such modified internucleoside linkages are often desired over the naturally occurring phosphodiester linkage because of advantageous properties they can impart such as, for example, enhanced cellular uptake, enhanced affinity for nucleic acid target and increased stability in the presence of nucleases.

Modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity can comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included. Representative U.S. patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050.

Another phosphorus containing modified internucleoside linkage is the phosphonomonoester (see U.S. Pat. Nos. 5,874,553 and 6,127,346). Phosphonomonoester nucleic acids have useful physical, biological and pharmacological properties in the areas of inhibiting gene expression (antisense oligonucleotides, ribozymes, sense oligonucleotides and triplex-forming oligonucleotides), as probes for the detection of nucleic acids and as auxiliaries for use in molecular biology.

Modified oligonucleotide backbones that do not include a phosphorus atom therein may have backbones that are formed for example, by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; acetyl, formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439.

Some additional examples of modified oligonucleotide backbones that do not contain a phosphorus atom therein include, —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). The MMI type and amide internucleoside linkages are disclosed in the below referenced U.S. Pat. Nos. 5,489,677 and 5,602,240, respectively.

The term “mixed backbone,” as used herein, refers to oligonucleotides containing at least two different types of internucleoside linkages.

Chimeric Oligomeric Compounds

It is not necessary for all positions in an oligomeric compound to be uniformly modified, and in fact more than one of the modifications described herein may be incorporated in a single oligomeric compound or even at a single monomeric subunit such as a nucleoside within an oligomeric compound. The present invention also includes oligomeric compounds which are chimeric oligomeric compounds. “Chimeric” oligomeric compounds or “chimeras,” in the context of this invention, are oligomeric compounds which contain two or more chemically distinct regions, each made up of at least one monomer unit, i.e., a nucleotide in the case of a nucleic acid based oligomer.

Chimeric oligomeric compounds typically contain at least one region modified so as to confer increased resistance to nuclease degradation, increased cellular uptake, and/or increased binding affinity for the target nucleic acid. An additional region of the oligomeric compound may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of inhibition of gene expression. Consequently, comparable results can often be obtained with shorter oligomeric compounds when chimeras are used, compared to for example phosphorothioate deoxyoligonucleotides hybridizing to the same target region. Cleavage of the RNA target can be routinely detected by gel electrophoresis and, if necessary, associated nucleic acid hybridization techniques known in the art.

Chimeric oligomeric compounds of the invention may be formed as composite structures of two or more oligonucleotides, oligonucleotide analogs, oligonucleosides and/or oligonucleotide mimetics as described herein. Such oligomeric compounds have also been referred to in the art as hybrids hemimers, gapmers, inverted gapmers or blockmers. Representative U.S. patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922.

Conjugates

Another substitution that can be appended to the oligomeric compounds of the invention involves the linkage of one or more moieties or conjugates which enhance the activity, cellular distribution or cellular uptake of the resulting oligomeric compounds. In one embodiment, such modified oligomeric compounds are prepared by covalently attaching conjugate groups to functional groups such as hydroxyl or amino groups. The term “conjugate group(s)” as used in the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196.

Conjugate moieties include but are not limited to lipophilic moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), athioether, e.g., hexyl-5-tritylthiol (Manoharan et al., Ann. N.Y. Acad. Sci., 1992, 660, 306-309; Manoharan et al., Bioorg. Med. Chem. Let., 1993, 3, 2765-2770), athiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylarrunonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

The oligomeric compounds of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130.

Representative U.S. patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Oligomeric compounds used in the compositions of the present invention can also be modified to have one or more stabilizing groups that are generally attached to one or both termini of oligomeric compounds to enhance properties such as for example nuclease stability. Included in stabilizing groups are cap structures. The terms “cap structure” or “terminal cap moiety,” as used herein, refer to chemical modifications, which have been incorporated at either terminus of oligonucleotides. These terminal modifications protect the oligomeric compounds having terminal nucleic acid moieties from exonuclease degradation, and can help in delivery and/or localization within a cell. The cap can be present at the 5′-terminus (5′-cap) or at the 3′-terminus (3′-cap) or can be present on both termini. In non-limiting examples, the 5′-cap includes inverted abasic residue (moiety), 4′,5′-methylene nucleotide; 1-(beta-D-erythrofuranosyl)nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl riucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

Particularly preferred 3′-cap structures of the present invention include, for example 4′,5′-methylene nucleotide; 1-(beta-D-erythrofuranosyl)nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Tyer, 1993, Tetrahedron 49, 1925).

Further 3′ and 5′-stabilizing groups that can be used to cap one or both ends of an oligomeric compound to impart nuclease stability include those disclosed in WO 03/004602.

Oligomer Mimetics

The terms “oligomer mimetic” and “oligonucleotide mimetic,” as used herein, refer to oligomeric compounds wherein the furanose ring and the internucleotide linkage are replaced with novel groups. The heterocyclic base moiety or modified heterocyclic base moiety is maintained for hybridization with an appropriate target nucleic acid. Such “oligomer mimetics” are often desired over the naturally occurring forms because of advantageous properties they can impart such as, for example, enhanced cellular uptake, enhanced affinity for nucleic acid target and increased stability in the presence of nucleases. Some non-limiting examples of “oligomer mimetics” are given below.

Replacing the sugar-backbone of an oligonucleotide with an amide containing backbone, results in peptide nucleic acids (PNA). The first PNA's reported (Nielsen et al., Science, 1991, 254, 1497-1500) consisted of nucleobases linked to the aza nitrogen atoms of the amide portion of an aminoethylglycine (aeg) backbone. These mimetics displayed favorable hybridization properties, high biological stability and are electrostatically neutral molecules. In one recent study PNA's were used to correct aberrant splicing in a transgenic mouse model (Sazani et al., Nat. Biotechnol., 2002, 20, 1228-1233). Since the first reports, numerous modifications have since been made to the basic PNA backbone, for example, incorporating a constrained cyclic aminoethylpropyl (aep) group, in place of the aeg group. Representative United States patents that teach the preparation of PNA oligomeric compounds include, but are not limited to, U.S. Pat. Nos. 5,539,082; 5,714,331; and 5,719,262. PNA's can be obtained commercially from Applied Biosystems (Foster City, Calif., USA).

Another class of oligonucleotide mimetic is based on nucleobases attached to linked morpholino units to form morpholino nucleic acid (MF). A number of linking groups have been reported that link the morpholino monomeric units in a morpholino nucleic acid. A preferred class of linking groups has been selected to give a non-ionic oligomeric compound, which are less likely to have undesired interactions with cellular proteins, (Dwaine A. Braasch and David R. Corey, Biochemistry, 2002, 41(14), 4503-4510). Morpholino-based oligomeric compounds are disclosed in U.S. Pat. Nos. 5,034,506, 5,166,315, and 5,185,444 and several studies on them have been reported (see: Genesis, volume 30, issue 3, 2001 and Heasman, J., Dev. Biol., 2002, 243, 209-214, and Nasevicius et al., Nat. Genet., 2000, 26, 216-220; and Lacerra et al., Proc. Natl. Acad. Sci., 2000, 97, 9591-9596).

A further class of oligonucleotide mimetic is cyclohexenyl nucleic acids (CeNA), whereby the sugar-backbone is replaced with a cyclohexenyl ring. CeNA DMT protected phosphoramidite monomers have been prepared and used for oligomeric synthesis using standard phosphoramidite chemistry. Fully modified cyclohexenyl nucleic acids and oligonucleotides having specific positions modified with CeNA have been prepared and studied (see Wang et al., J. Am. Chem. Soc., 2000, 122, 8595-8602). In general, the incorporation of CeNA monomers into a DNA chain increases its stability in DNA/RNA hybrids, and was shown by NMR and circular dichroism to proceed with easy conformational adaptation. CeNA oligoadenylates formed complexes with RNA and DNA complements with similar stability to the native complexes. Furthermore, a sequence targeting RNA that incorporated CeNA, was stable to serum and able to activate E. Coli RNase resulting in cleavage of the target RNA strand.

Another class of oligonucleotide mimetic (anhydrohexitol nucleic acid) can be prepared from one or more anhydrohexitol nucleosides (see, Wouters and Herdewijn, Bioorg. Med. Chem. Lett., 1999, 9, 1563-1566). The above oligonucleotide mimetics can be considered as repeating units of the monomers depicted below:

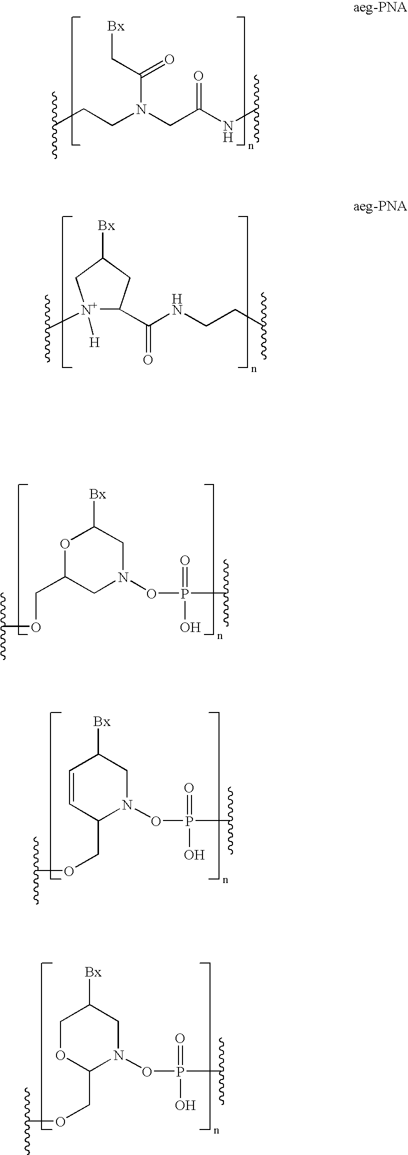

wherein,

each Bx is independently a nucleobase,

n is from 2 to about 50, and

[symbol] represents connection to the next repeating monomer, or end terminus.

Oligomer Synthesis

Oligomerization of modified and unmodified nucleosides is performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713) synthesis as appropriate. In addition specific protocols for the synthesis of oligomeric compounds of the invention are illustrated in the examples below.

Oligonucleotides are generally prepared either in solution or on a support medium, e.g. a solid support medium. In general a first synthon (e.g. a monomer, such as a nucleoside) is first attached to a support medium, and the oligonucleotide is then synthesized by sequentially coupling monomers to the support-bound synthon. This iterative elongation eventually results in a final oligomeric compound or other polymer such as a polypeptide. Suitable support medium can be soluble or insoluble, or may possess variable solubility in different solvents to allow the growing support bound polymer to be either in or out of solution as desired. Traditional support medium such as solid support media are for the most part insoluble and are routinely placed in reaction vessels while reagents and solvents react with and/or wash the growing chain until the oligomer has reached the target length, after which it is cleaved from the support and, if necessary further worked up to produce the final polymeric compound. More recent approaches have introduced soluble supports including soluble polymer supports to allow precipitating and dissolving the iteratively synthesized product at desired points in the synthesis (Gravert et al., Chem. Rev., 1997, 97, 489-510).

The terms “support medium,” “solid support,” or “solid support medium” are intended to include all forms of support known to one of ordinary skill in the art for the synthesis of oligomeric compounds and related compounds such as peptides. Some representative support medium that are amenable to the methods of the present invention include but are not limited to the following: controlled pore glass (CPG); oxalyl-controlled pore glass (see, e.g., Alul, et al., Nucleic Acids Research 1991, 19, 1527); silica-containing particles, such as porous glass beads and silica gel such as that formed by the reaction of trichloro-[3-(4-chloromethyl)phenyl]propylsilane and porous glass beads (see Parr and Grohmann, Angew. Chem. Internal Ed. 1972, 11, 314, sold under the trademark “PORASIL E” by Waters Associates, Framingham, Mass., USA); the mono ester of 1,4-dihydroxymethylbenzene and silica (see Bayer and Jung, Tetrahedron Lett., 1970, 4503, sold under the trademark “BIOPAK” by Waters Associates); TENTAGEL (see, e.g., Wright, et al., Tetrahedron Letters 1993, 34, 3373); cross-linked styrene/divinylbenzene copolymer beaded matrix or POROS, a copolymer of polystyrene/divinylbenzene (available from Perceptive Biosystems); soluble support medium, polyethylene glycol PEG's (see Bonora et al., Organic Process Research & Development, 2000, 4, 225-231).

Further support medium amenable to the present invention include without limitation PEPS support a polyethylene (PE) film with pendant long-chain polystyrene (PS) grafts (molecular weight on the order of 106, (see Berg, et al., J. Am. Chem. Soc., 1989, 111, 8024 and International Patent Application WO 90/02749),). The loading capacity of the film is as high as that of a beaded matrix with the additional flexibility to accommodate multiple syntheses simultaneously. The PEPS film may be fashioned in the form of discrete, labeled sheets, each serving as an individual compartment. During all the identical steps of the synthetic cycles, the sheets are kept together in a single reaction vessel to permit concurrent preparation of a multitude of peptides at a rate close to that of a single peptide by conventional methods. Also, experiments with other geometries of the PEPS polymer such as, for example, non-woven felt, knitted net, sticks or microwellplates have not indicated any limitations of the synthetic efficacy.

Further support medium amenable to the present invention include without limitation particles based upon copolymers of dimethylacrylamide cross-linked with N,N′-bisacryloylethylenediamine, including a known amount of N-tertbutoxycarbonyl-beta-alanyl-N′-acryloylhexamethylenediamine. Several spacer molecules are typically added via the beta alanyl group, followed thereafter by the amino acid residue subunits. Also, the beta alanyl-containing monomer can be replaced with an acryloyl safcosine monomer during polymerization to form resin beads. The polymerization is followed by reaction of the beads with ethylenediamine to form resin particles that contain primary amines as the covalently linked functionality. The polyacrylamide-based supports are relatively more hydrophilic than are the polystyrene-based supports and are usually used with polar aprotic solvents including dimethylformamide, dimethylacetamide, N-methylpyrrolidone and the like (see Atherton, et al., J. Am. Chem. Soc., 1975, 97, 6584, Bioorg Chem. 1979, 8, 351, and J. C. S. Perkin I 538 (1981)).

Further support medium amenable to the present invention include without limitation a composite of a resin and another material that is also substantially inert to the organic synthesis reaction conditions employed. One exemplary composite (see Scott, et al., J. Chrom. Sci., 1971, 9, 577) utilizes glass particles coated with a hydrophobic, cross-linked styrene polymer containing reactive chloromethyl groups, and is supplied by Northgate Laboratories, Inc., of Hamden, Conn., USA. Another exemplary composite contains a core of fluorinated ethylene polymer onto which has been grafted polystyrene (see Kent and Merrifield, Israel J. Chem. 1978, 17, 243 and van Rietschoten in Peptides 1974, Y. Wolman, Ed., Wiley and Sons, New York, 1975, pp. 113-116). Contiguous solid support media other than PEPS, such as cotton sheets (Lebl and Eichler, Peptide Res. 1989, 2, 232) and hydroxypropylacrylate-coated polypropylene membranes (Daniels, et al., Tetrahedron Lett. 1989, 4345). Acrylic acid-grafted polyethylene-rods and 96-microtiter wells to immobilize the growing peptide chains and to perform the compartmentalized synthesis. (Geysen, et al., Proc. Natl. Acad. Sci. USA, 1984, 81, 3998). A “tea bag” containing traditionally-used polymer beads. (Houghten, Proc. Natl. Acad. Sci. USA, 1985, 82, 5131). Simultaneous use of two different supports with different densities (Tregear, Chemistry and Biology of Peptides, J. Meienhofer, ed., Ann Arbor Sci. Publ., Ann Arbor, 1972 pp. 175-178). Combining of reaction vessels via a manifold (Gorman, Anal. Biochem., 1984, 136, 397). Multicolumn solid-phase synthesis (e.g., Krchnak, et al., Int. J. Peptide Protein Res., 1989, 33, 209), and Holm and Meldal, in “Proceedings of the 20th European Peptide Symposium”, G. Jung and E. Bayer, eds., Walter de Gruyter & Co., Berlin, 1989 pp. 208-210). Cellulose paper (Eichler, et al., Collect. Czech. Chem. Commun., 1989, 54, 1746). Support mediumted synthesis of peptides have also been reported (see, Synthetic Peptides: A User's Guide, Gregory A. Grant, Ed. Oxford University Press 1992; U.S. Pat. Nos. 4,415,732; 4,458,066; 4,500,707; 4,668,777; 4,973,679; 5,132,418; 4,725,677 and Re-34,069.)

Support bound oligonucleotide synthesis relies on sequential addition of nucleotides to one end of a growing chain. Typically, a first nucleoside (having protecting groups on any exocyclic amine functionalities present) is attached to an appropriate glass bead support and nucleotides bearing the appropriate activated phosphite moiety, i.e. an “activated phosphorous group” (typically nucleotide phosphoramidites, also bearing appropriate protecting groups) are added stepwise to elongate the growing oligonucleotide. Additional methods for solid-phase synthesis may be found in Caruthers U.S. Pat. Nos. 4,415,732; 4,458,066; 4,500,707; 4,668,777; 4,973,679; and 5,132,418; and Koster U.S. Pat. Nos. 4,725,677 and Re. 34,069.

Commercially available equipment routinely used for the support medium based synthesis of oligomeric compounds and related compounds is sold by several vendors including, for example, Applied Biosystems (Foster City, Calif.). Any other means for such synthesis known in the art may additionally or alternatively be employed. Suitable solid phase techniques, including automated synthesis techniques, are described in F. Eckstein (ed.), Oligonucleotides and Analogues, a Practical Approach, Oxford University Press, New York (1991).

The term “linking moiety,” as used herein is generally a di-functional group, covalently binds the ultimate 3′-nucleoside (and thus the nascent oligonucleotide) to the solid support medium during synthesis, but which is cleaved under conditions orthogonal to the conditions under which the 5′-protecting group, and if applicable any 2′-protecting group, are removed. Suitable linking moietys include, but are not limited to, a divalent group such as alkylene, cycloalkylene, arylene, heterocyclyl, heteroarylene, and the other variables are as described above. Exemplary alkylene linking moietys include, but are not limited to, C₁-C₁₂ alkylene (e.g. preferably methylene, ethylene (e.g. ethyl-1,2-ene), propylene (e.g. propyl-1,2-ene, propyl-1,3-ene), butylene, (e.g. butyl-1,4-ene, 2-methylpropyl-1,3-ene), pentylene, hexylene, heptylene, octylene, decylene, dodecylene), etc. Exemplary cycloalkylene groups include C₃-C₁₂ cycloalkylene groups, such as cyclopropylene, cyclobutylene, cyclopentanyl-1,3-ene, cyclohexyl-1,4-ene, etc. Exemplary arylene linking moietys include, but are not limited to, mono- or bicyclic arylene groups having from 6 to about 14 carbon atoms, e.g. phenyl-1,2-ene, naphthyl-1,6-ene, napthyl-2,7-ene, anthracenyl, etc. Exemplary heterocyclyl groups within the scope of the invention include mono- or bicyclic aryl groups having from about 4 to about 12 carbon atoms and about 1 to about 4 hetero atoms, such as N, O and S, where the cyclic moieties may be partially dehydrogenated. Certain heteroaryl groups that may be mentioned as being within the scope of the invention include: pyrrolidinyl, piperidinyl (e.g. 2,5-piperidinyl, 3,5-piperidinyl), piperazinyl, tetrahydrothiophenyl, tetrahydrofuranyl, tetrahydro quinolinyl, tetrahydro isoquinolinyl, tetrahydroquinazolinyl, tetrahydroquinoxalinyl, etc. Exemplary heteroarylene groups include mono- or bicyclic aryl groups having from about 4 to about 12 carbon atoms and about 1 to about 4 hetero atoms, such as N, O and S. Certain heteroaryl groups that may be mentioned as being within the scope of the invention include: pyridylene (e.g. pyridyl-2,5-ene, pyridyl-3,5-ene), pyrimidinyl, thiophenyl, furanyl, quinolinyl, isoquinolinyl, quinazolinyl, quinoxalinyl, etc.

Suitable reagents for introducing the group HOCO-Q-CO include diacids (HO₂C-Q-CO₂H). Particularly suitable diacids include malonic acid (Q is methylene), succinic acid (Q is 1,2-ethylene), glutaric acid, adipic acid, pimelic acid, and phthalic acid. Other suitable reagents for introducing HOCO-Q-CO above include diacid anhydrides. Particularly suitable diacid anhydrides include malonic anhydride, succinic anhydride, glutaric anhydride, adipic anhydride, pimelic anhydride, and phthalic anhydride. Other suitable reagents for introducing HOCO-Q-CO include diacid esters, diacid halides, etc. One especially preferred reagent for introducing HOCO-Q-CO is succinic anhydride.

The compound of formula may be linked to a support via terminal carboxylic acid of the HOCO-Q-CO group, via a reactive group on the support medium. In some embodiments, the terminal carboxylic acid forms an amide linkage with an amine reagent on the support surface. In other embodiments, the terminal carboxylic acid forms an ester with an OH group on the support medium. In some embodiments, the terminal carboxylic acid may be replaced with a terminal acid halide, acid ester, acid anhydride, etc. Specific acid halides include carboxylic chlorides, bromides and iodides. Specific esters include methyl, ethyl, and other C₁-C₁₀ alkyl esters. Specific anhydrides include formyl, acetyl, propanoyl, and other C₁-C₁₀ alkanoyl esters.

The present invention also encompasses the preparation of oligomeric compounds incorporating at least one 2′-O-protected nucleoside into the oligomeric compounds delineated herein. After incorporation and appropriate deprotection the 2′-O-protected nucleoside will be converted to a ribonucleoside at the position of incorporation. The number and position of the 2-ribonucleoside units in the final oligomeric compound can vary from one at any site or the strategy can be used to prepare up to a full 2′-OH modified oligomeric compound. All 2′-protecting groups amenable to the synthesis of oligomeric compounds are included in the present invention. In general, a protected nucleoside is attached to a solid support by for example a succinate linker. Then the oligonucleotide is elongated by repeated cycles of deprotecting the 5′-terminal hydroxyl group, coupling of a further nucleoside unit, capping and oxidation (alternatively sulfurization). In a more frequently used method of synthesis the completed oligonucleotide is cleaved from the solid support with the removal of phosphate protecting groups and exocyclic amino protecting groups by treatment with an ammonia solution. Then a further deprotection step is normally required for the more specialized protecting groups used for the protection of 2′-hydroxyl groups which will give the fully deprotected oligonucleotide.

A large number of 2′-protecting groups have been used for the synthesis of oligoribonucleotides but over the years more effective groups have been discovered. The key to an effective 2′-protecting group is that it is capable of selectively being introduced at the 2′-position and that it can be removed easily after synthesis without the formation of unwanted side products. The protecting group also needs to be inert to the normal deprotecting, coupling, and capping steps required for oligoribonucleotide synthesis. Some of the protecting groups used initially for oligoribonucleotide synthesis included tetrahydropyran-1-yl and 4-methoxytetrahydropyran-4-yl. These two groups are not compatible with all 5′-protecting groups so modified versions were used with 5′-DMT groups such as 1-(2-fluorophenyl)-4-methoxypiperidin-4-yl (Fpmp). Reese has identified a number of piperidine derivatives (like Fpmp) that are useful in the synthesis of oligoribonucleotides including 1-[(chloro-4-methyl)phenyl]-4′-methoxypiperidin-4-yl (Reese et al., Tetrahedron Lett., 1986, (27), 2291). Another approach was to replace the standard 5′-DMT (dimethoxytrityl) group with protecting groups that were removed under non-acidic conditions such as levulinyl and 9-fluorenylmethoxycarbonyl. Such groups enable the use of acid labile 2′-protecting groups for oligoribonucleotide synthesis. Another more widely used protecting group initially used for the synthesis of oligoribonucleotides was the t-butyldimethylsilyl group (Ogilvie et al., Tetrahedron Lett., 1974, 2861; Hakimelahi et al., Tetrahedron Lett., 1981, (22), 2543; and Jones et al., J. Chem. Soc. Perkin I., 2762). The 2′-O-protecting groups can require special reagents for their removal such as for example the t-butyldimethylsilyl group is normally removed after all other cleaving/deprotecting steps by treatment of the oligomeric compound with tetrabutylammonium fluoride (TBAF).

One group of researchers examined a number of 2′-protecting groups (Pitsch, S., Chimia, 2001, (55), 320-324.) The group examined fluoride labile and photolabile protecting groups that are removed using moderate conditions. One photolabile group that was examined was the [2-(nitrobenzyl)oxy]methyl (nbm) protecting group (Schwartz et al., Bioorg. Med. Chem. Lett., 1992, (2), 1019.) Other groups examined included a number structurally related formaldehyde acetal-derived, 2′-protecting groups. Also prepared were a number of related protecting groups for preparing 2′-O-alkylated nucleoside phosphoramidites including 2′-O—[(triisopropylsilyl)oxy]methyl (2′-O—CH₂—O—Si(iPr)₃, TOM). One 2′-protecting group that was prepared to be used orthogonally to the TOM group was 2′-O—[(R)-1-(2-nitrophenyl)ethyloxy)methyl] ((R)-mnbm).

Another strategy using a fluoride labile 5′-protecting group (non-acid labile) and an acid labile 2′-protecting group has been reported (Scaringe, Stephen A., Methods, 2001, (23) 206-217). A number of possible silyl ethers were examined for 5′-protection and a number of acetals and orthoesters were examined for 2′-protection. The protection scheme that gave the best results was 5′-O-silyl ether-2′-ACE (5′-O-bis(trimethylsiloxy)cyclododecyloxysilyl ether (DOD)-2′-O-bis(2-acetoxyethoxy)methyl (ACE). This approach uses a modified phosphoramidite synthesis approach in that some different reagents are required that are not routinely used for RNA/DNA synthesis.

Although a lot of research has focused on the synthesis of oligoribonucleotides the main RNA synthesis strategies that are presently being used commercially include 5′-O-DMT-2′-O-t-butyldimethylsilyl (TBDMS), 5′-O-DMT-2′-O-[1(2-fluorophenyl)-4-methoxypiperidin-4-yl] (FPMP), 2′-O—[(triisopropylsilyl)oxy]methyl (2′-O—CH₂—O—Si(iPr)₃ (TOM), and the 5′-O-silyl ether-2′-ACE (5′-O-bis(trimethylsiloxy)cyclododecyloxysilyl ether (DOD)-2′-O-bis(2-acetoxyethoxy)methyl (ACE). A current list of some of the major companies currently offering RNA products include Pierce Nucleic Acid Technologies, Dharmacon Research Inc., Ameri Biotechnologies Inc., and Integrated DNA Technologies, Inc. One company, Princeton Separations, is marketing an RNA synthesis activator advertised to reduce coupling times especially with TOM and TBDMS chemistries. Such an activator would also be amenable to the present invention. The primary groups being used for commercial RNA synthesis, include, but are not limited to:
TBDMS=5′-O-DMT-2′-O-t-butyldimethylsilyl;
TOM=2′-O-[(triisopropylsilyl)oxy]methyl;
DOD/ACE=5′-O-bis(trimethylsiloxy)cyclododecyloxysilylether-2′-O-bis(2-acetoxyethoxy)methyl;
FPMP=5′-O-DMT-2′-O-[1(2-fluorophenyl)-4-methoxypiperidin-4-yl]; or
CPEP=2′-O-[1(4-chlorophenyl)-4-ethoxypiperidin-4-yl].

All of the aforementioned RNA synthesis strategies are amenable to the present invention. Strategies that would be a hybrid of the above e.g. using a 5′-protecting group from one strategy with a 2′-protecting from another strategy is also amenable to the present invention.

All of the aforementioned RNA synthesis strategies are amenable to the present invention. Strategies that would be a hybrid of the above e.g. using a 5′-protecting group from one strategy with a 2′-protecting from another strategy is also amenable to the present invention.

The preparation of ribonucleotides and oligomeric compounds having at least one ribonucleoside incorporated and all the possible configurations falling in between these two extremes are encompassed by the present invention.

Synthetic Methods

The 9-phenylxanthyl (pixyl) group was introduced by Colin Reese in 1978 as an alternative protecting group to dimethoxytrityl (DMT) group (Chattopadhyaya, J. B.; Reese, C. B.; J. Chem SOC. Chem. Comm. (1978) 639-40). The pixyl group has a similar stability towards acids to DMT group. In general, pixyl protected nucleosides are more likely to be crystalline. The reagent for putting a pixyl group on a 6 hydroxyl function is 9-chloro-9-phenylxanthene (pixyl chloride). The synthesis of pixyl chloride was achieved via reacting xanthone with phenyl magnesium bromide to give 9-phenylxanthenol which is then chlorinated with acetic chloride to afford pixyl chloride. The starting materials for the synthesis of pixyl chloride are expensive and the Grignard reagent is hazardous which limited widespread use of this reagent for oligonucleotide synthesis.

An additional embodiment of the present invention are new routes, through a Friedel-Craft reaction, of synthesizing pixyl analogs. A diaryl ether is reacted with an α,α,α-trichlorotoluene in the presence of an acid as a catalyst. The ortho- and para-positions of the ether are the reactive sites. In order to improve the yield and simplify the purification procedures, it is favorable and preferred to use para-substituents on the ether to block the undesired reactive sites. This reaction is also applied to diaryl thioether and diaryl amines. Substitutions at the meta positions of the toluene or the diaryl ether also can be incorporated to adjust the electronic reactivity of the final pixyl group. The α,α,α-trichlorotoluene can be replaced with the corresponding aryl acid, aryl acid ester, aryl acid chloride, aryl cyamide and aryl amide. The catalyst can be any of the Friedel Crafts acids, preferably zinc chloride and aluminum chloride. The starting materials for this route are widely accessible, inexpensive and non-hazardous. The yields of the key step, Friedel-Crafts reaction, can be as high as over 90% (see examples).

In a preferred synthetic route, the substituted pixyl chloride can be prepared in 90% yield from the appropriately substituted phenyl ether and an aromatic carboxylic acid. The substituted pixyl nucleosides are crystalline compounds, which facilitates their purification without chromatography.
General Procedure for Synthesis of Pixyl Analog (as the Alcohols)

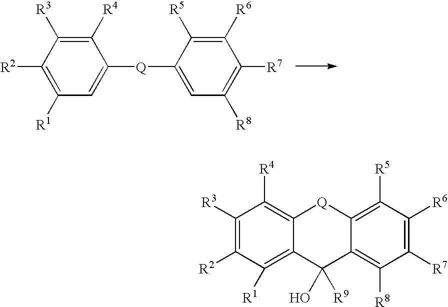

To a stirred mixture of substituted or unsubstituted diphenylether (1.01 mole), ring substituted or unsubstituted benzoic acid (HOOCR⁹ where R⁹ is phenyl) (1.13 mole) and anhydrous zinc chloride (400 g; 2.94 mole) is added phosphorousoxy trichloride (300 mL; 3.27 mole) slowly using an addition funnel. The reaction mixture is then slowly heated to 95° C. after the reaction starts and is monitored by tlc. After the reaction is complete, ethyl acetate (500 mL) is added, followed by water (200 mL) slowly. An additional amount of water (2500 mL) is added at a faster rate. The mixture is stirred overnight at room temperature and a solid will come out of the solution. The solid is filtered and recrystallized from methanol to afford the substituted pixyl alcohol product.

Conversion of the Pixyl Analogs from Alcohols to Reactive Alkyl Halides

To a stirred solution of the substituted pixyl alcohol (0.982 mole) in dichloromethane (1000 mL) is added thionyl chloride (102 ml; 1.1 mole) slowly with cooling. The reaction is monitored by thin layer chromatography. When complete, the reaction is concentrated, toluene added followed by hexane to afford the pixyl analog as the chloride.

### EXAMPLES

The present invention may be further appreciated upon reference to the following, non-limiting examples.

### Example 1

### Synthesis of 2,7-Dimethyl-9-phenylxanthen-9-ol (DMPx-OH)

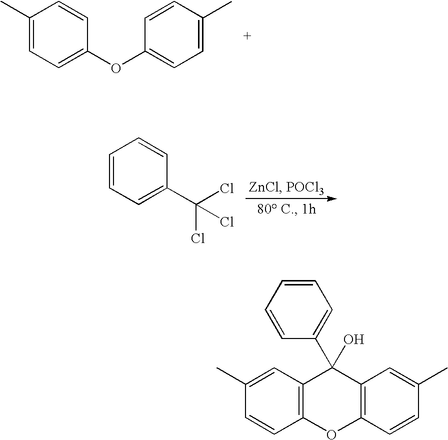

Tolyl ether (20 g, 0.10 mol), α,α,α-trichlorotoluene (20 ml, 0.12 mol), zinc chloride (40 g, 0.29 mol) and phosphorus oxychloride (30 ml, 0.32 mol) were heated at 84° C. for 1 hour. The mixture was cooled to room temperature and poured into water (500 ml). The flask was rinsed with ethyl acetate (50 ml) and the suspension was stirred overnight. The mixture was then filtered, washed with water and methanol and dried to give the crude title compound as a solid.

### Example 2

### Alternate synthesis of 2,7-Dimethyl-9-phenylxanthen-9-ol (DMPx-OH)

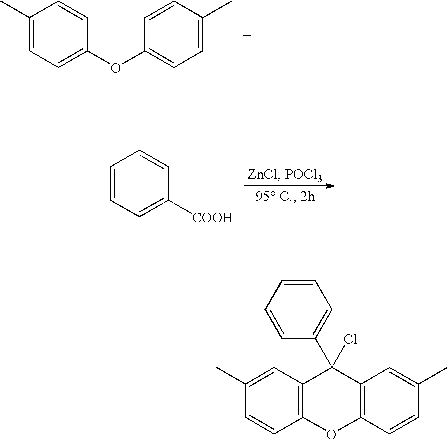

Tolyl ether (10 g, 0.05 mol), benzoic acid (7.5 g, 0.06 mol), zinc chloride (20 g, 0.15 mol) and phosphorus oxychloride (15 ml, 0.16 mol) were heated at 95° C. for two hours. The mixture was cooled to room temperature and ethyl acetate (25 ml) was added to form a suspension. The suspension was poured into 500 ml stirring DI water at room temperature. The mixture was heated under reflux for 15 minutes and cooled down to room temperature overnight. The mixture was filtered and washed with water (100 ml). The damp cake was suspended with 300 ml of methanol and stirred to boil for 2 or 3 minutes. The resultant suspension was allowed to cool to room temperature over a period of 3 hrs and was then filtered, washed with methanol and dried to give the title compound as a solid (14 g, 91.8%).

### Example 3

### Synthesis of 9-Chloro-2,7-Dimethyl-9-phenylxanthene (DMPx-Cl)

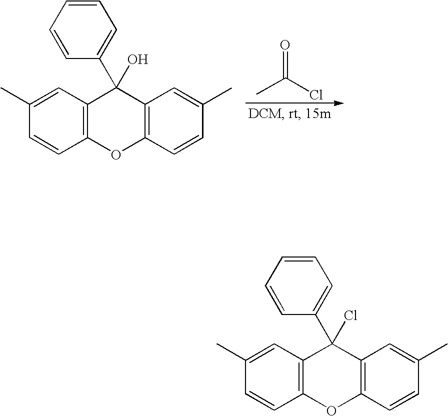

Acetyl chloride (1 ml) was added to a solution of DMPx-OH (1 g) in methylene chloride (10 ml). The mixture was stirred at room temperature for 15 min and the solvent removed under reduce pressure. The residue was stirred with n-hexane (200 ml) at room temperature. The solid was filtered and washed with n-hexane to give the title product (0.8 g, 79%).

### Example 4

### 2,7-Bromo-9-phenylxanthen-9-ol

Bis-(4-bromophenyl)ether (30 g, 0.092 mol), α,α,α-trichlorotoluene (22 ml, 0.15 mol), aluminum chloride (20 g, 0.15 mol) in dichloromethane (75 ml) were stirred at room temperature for 1 hour. The reaction mixture was poured into water (100 ml) and hexane (300 ml and the suspension was stirred overnight. The mixture was then filtered, washed with water (230 ml) and hexane 400 ml) and dried to give the title compound as a crystalline solid (34.94 g, yield: 88%).

### Example 5

### 5′-DMPx-thymidine

Thymidine (2.4 g, 10 mmol) was dissolved in pyridine (15 ml) and DMPx-Cl (4.1 g, 11.5 mmol) was added. The mixture was stirred at room temperature for 30 min. The mixture was diluted with ethyl acetate (50 ml) and washed with water (2×50 ml). The mixture was evaporated to dryness and the solid was dissolved in dichloromethane (15 ml). Hexane (50 ml) was added and the mixture was stirred overnight. Filtration gave the title compound as a solid (4.44 g, 79%).

### Example 6

### Synthesis of 2,7-Dimethyl-9-(4-t-Butyl)Phenylxanthene-9-Ol (t-But-DMPx)

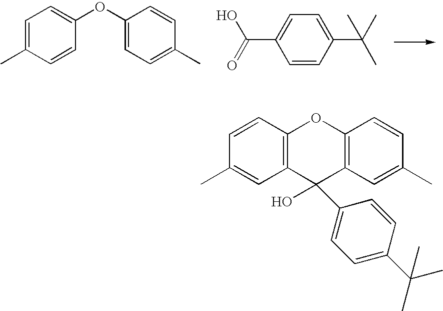

Tolyl ether (200 g, 1.01 moles), t-butylbenzoic acid (201 g, 1.13 moles), zinc chloride (400 g, 2.9 moles) and phosphorus oxychloride (300 ml, 3.2 moles) were stirred at 95 degree in an oil bath for 2 hrs. The mixture was cooled to room temperature and ethyl acetate (500 ml) was added. The suspension was stirred with water (3 liters) overnight. The solid was filtered, washed with water and n-hexane. After drying overnight, the titled compound was collected (294 g, yield: 81%).

### Example 7

### Synthesis of 2,7-Dimethyl-9-Biphenylxanthene-9-Ol (BipheDMPx-OH)

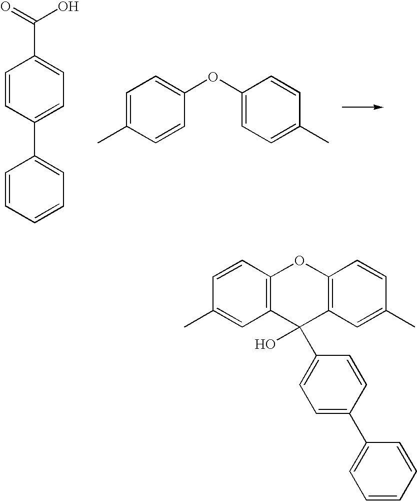

Tolyl Ether (5.9 gm, 30 mmoles), bipheylcarboxylic acid (6 gm, 30.27 mmoles), zinc chloride (12 gm, 88 mmoles) and phosphorus oxychloride (20 mmoles) were stirred at 95 degree in an oil bath for 2 hrs. The mixture was cooled to room temperature and the viscous mixture is poured into cracked ice and stirred overnight. The solid was collected and washed with water. The solid was suspended in 150 ml of methanol and was heated to boiling for 5 min. The mixture was cooled to room temperature, filtered and dried to a constant weight (8.4 g, yield: 74%).

### Example 8

### Synthesis of 2,7-Di-t-Butyl-9-Phenylxanthene-9-ol (D-tBut-Px-OH)

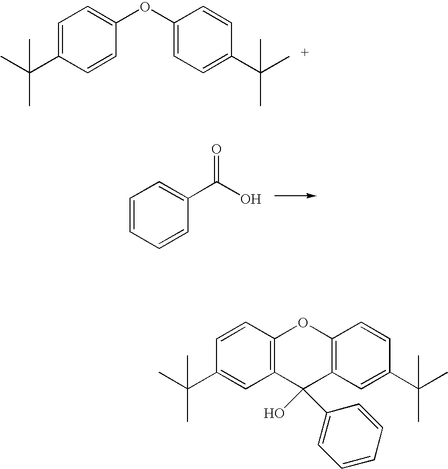

t-Butylphenylether (7.49 gm, 26.52 mmol), benzoic acid (3.24 gm, 26.52 mmol), zinc chloride (10 gm, 79.56 mmoles) and phosphorus oxychloride (12 ml, 132 mmoles) were stirred at 95 degree in an oil bath for 1.5 hrs. The mixture was cooled to room temperature and methanol (10 ml), ethylacetate (10 ml) and water (100 ml) were added. After stirring at room temperature overnight, the product was extracted into ethyl acetate. The upper phase was washed with 1N aqueous NaOH and water and distilled under reduced pressure. After silica-gel purification, the title compound was obtained (4 g, yield: 39%).

### Example 9

### 2,7-Dimethyl-9-Orthomethyl-Thiophenylxanthene-9-ol (DMTPx)

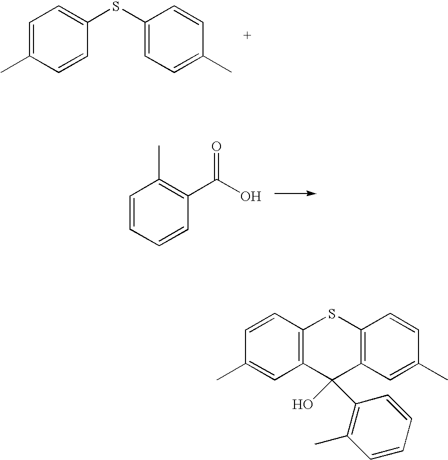

Ditolylthioether (2 gm, 9.33 mmoles), 2-methylbenzoic acid (1.36 gm, 9.98 mmoles), zinc chloride (4 gm, 29.35 mmoles) and phosphorus oxychloride (3 ml, 32.75 mmoles) were stirred at 95 degree in an oil bath for 2 hrs. The mixture was cooled to room temperature. Ethyl acetate and water (100 ml each) were added. The upper phase was washed twice with water and stripped to an oily solid. The title compound was treated with hot methanol, cool and filtered (0.75 g, yield: 25%).

### Example 10

### Synthesis of 9-chloro-2,7-Dimethyl-9-Phenylxanthene (DMPx-Cl)

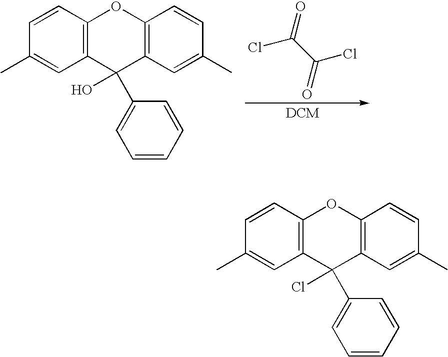

Oxalylchloride (23 ml, 0.27 moles) was added to a stirring solution of DMPx-OH (135 gm, 0.45 moles) in 250 ml of dichloromethane over 10 minutes period. After 30 min. the solution was evaporated under reduced pressure to a solid. The residue was treated with hexane, filtered and washed with hexane to give the title product (130 g, yield: 90%).

### Example 11

### Synthesis of 5′-DMPx-2′-methoxyethyl-5-methyl-N-benzoylcytidine

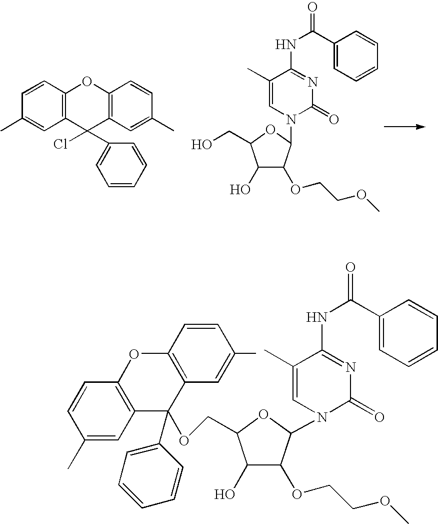

The mixture of 2′-methoxyethyl-5-methyl-N-benzoylcytidine (30 gm, 0.0715 moles), DMF (150 ml) and lutidine (21 ml, 0.172 moles) was stirred at room temperature. DMPx-chloride (26 gm, 0.0786 moles) was added in three portions over a 30 min. After 2 hours, ethyl acetate (700 ml) was added. The mixture was washed with saturated sodium bicarbonate, water and saturated sodium chloride. The upper layer was distilled under reduced pressure and the residue was purified by silica gel chromatography to give the title compound (36 g, yield: 79%).

### Example 12

### 5′-DMPx-2′-methoxyethyl-methyl-N-benzoylcytidine-3′-phosphoramidite

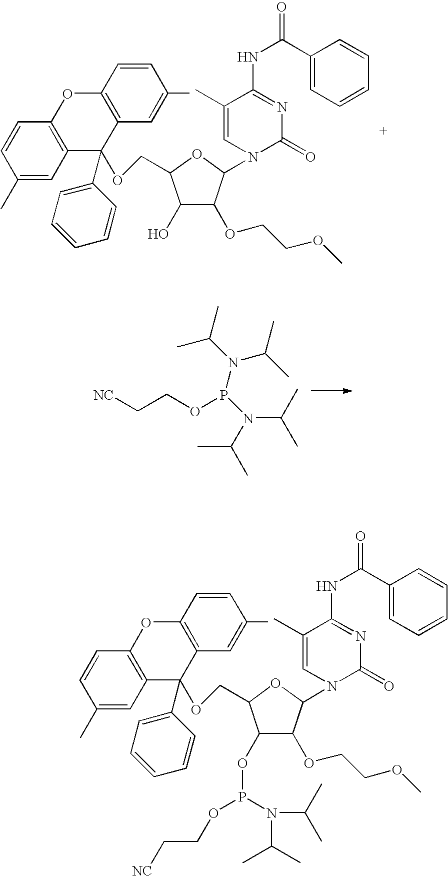

2-Cyanoethyl tetraisopropylphosphorodiamidite (60 ml, 71.46 mmole) was added to the stirred mixture of 5′-DMPx-2′-methoxyethyl-5-Methyl-N-benzoylcytidine (30 gm, 47.64; mmoles) at room temperature. After 3 min, tetrazole (2.6 gm, 38.11 mmoles) and 1-methylimidazole (0.4 ml, 4.76 mmoles) were added. After stirring for 1.5 hrs, triethylamine (7 ml), water (20 ml), DMF (70 ml) and hexane (40 ml) were added followed by a phase separation. The lower phase was washed with 2×50 ml of extracted with hexane. Then the product was isolated by silica gel chromatography to give the title compound (26.28 g, yield: 61%).

### Example 13

### Triethylammonium 5′-O-DMPx-thymidine 3′-H-phosphonate

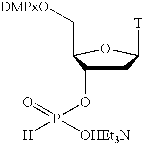

Ammonium phenyl H-phosphonate (5.25 g, 30 mmol), 5′-O-DMPx-thymidine (5.4 g, 10 mmol) and triethylamine (8.4 ml, 60 mmol) in pyridine (50 ml) were evaporated together under reduced pressure. The residue was coevaporated with dry pyridine (50 ml). The residue was dissolved in dry pyridine (50 ml) and the solution was cooled to 0° C. Pivaloyl chloride (3.7 ml, 30 mmol) was added dropwise over 10 min. After 30 min at 0° C., water (10 ml) was added and the stirred mixture was allowed to warm up to room temperature. Potassium phosphate buffer (1.0 M, pH 7.0, 250 ml) was added and the resulting mixture was concentrated under reduced pressure until all pyridine was removed. The residue was partitioned between dichloromethane (250 ml) and water (200 ml). The organic layer was washed with triethylammonium phosphate buffer (0.5 m, pH 7, 3×100 ml) and then evaporated. The residue was purified by a short silica gel column, eluted with dichloromethane-methanol (95:5 to 90:10). Evaporation of appropriate fractions to give the desired product (7.1 g).

A person of ordinary skill in the art will recognize that further embodiments are possible within the general scope of the foregoing description and the attached drawings and claims, and it would be within the skill of such skilled person to practice the invention as generally described herein. All references cited herein are expressly incorporated herein by reference.

1. A compound of formula I: 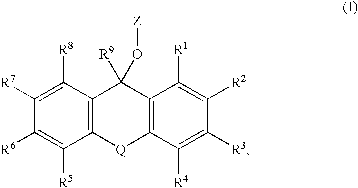
wherein:
R¹, R³, R⁴, R⁵, R⁶ and R⁸ are each, independently, H or alkyl or substituted alkyl;
R² and R⁷ are each, independently, alkyl, substituted alkyl, alkenyl, substituted alkenyl, alkynyl, substituted alkynyl, aryl, substituted aryl, hydroxyl, chloro, floro, iodo, cyano, azido, nitro, —C(═O)O—R¹⁰, —O—C(═O)—R¹⁰, —C(═O)N(R¹⁰)R¹¹, —N(R¹⁰)C(═O)R¹¹, —N(R¹⁰)R¹¹, —O—R¹⁰, or —S—R¹⁰;
or two or more groups R¹-R⁸, together with the ring carbons to which they are attached, combine to form a cyclic moiety selected from substituted or unsubstituted alicyclic, substituted or unsubstituted heterocyclic, substituted or unsubstituted aromatic, or substituted or unsubstituted heteroaromatic;
R⁹ is alkyl, substituted alkyl, alkenyl, substituted alkenyl, alkynyl, substituted alkynyl, aryl or substituted aryl;
R¹⁰ is H or alkyl;
R¹¹ is H or alkyl;
Z is a deoxy residue of a protected compound selected from a nucleoside, a nucleotide, a solid support-bound nucleotide, a nucleotide phosphoroamidite, an oligonucleotide, an oligonucleotide blockmer, or a solid support-bound oligonucleotide; and
Q is O, S, NR¹⁰, N(C═O)R¹⁰.

2. A compound of claim 1, wherein R¹, R³, R⁴, R⁵, R⁶ and R⁸ are each H.

3. A compound of claim 2, wherein R² and R⁷ are selected from alkyl or substituted alkyl.

4. A compound of claim 1, wherein any one of the protected compounds comprises at least one modified sugar, a 2′-substituent, or a conjugate group.

5. A compound of claim 4, wherein the 2′-substituent is selected from fluoro, alkoxy, substituted alkoxy, or OPR, wherein PR is a 2′-protecting group.

6. A compound of claim 5, wherein the 2′-substituent is selected from fluoro, OCH₃, OCH₂CH₂OCH₃, or OCH₂CH₂ON(CH₃)₂.

7. A compound of claim 5, wherein the 2′-substitutent is OPR.

8. A compound of claim 7, wherein PR is selected from CPEP, ACE, TOM, TBDMS, or Fpmp.

9. A compound of claim 4, wherein the modified sugar is a locked nucleic acid, or a 4′-thio nucleic acid.

10. A compound of claim 4, wherein the conjugate group comprises a lipophilic moiety.

11. A compound of claim 10, wherein the lipophilic moiety is selected from a cholesterol moiety or a polyethylene glycol moiety.

12. A compound of formula (II): 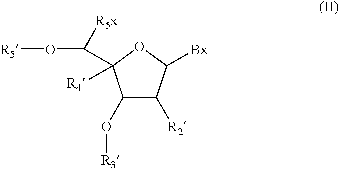
wherein
Bx is an optionally protected heterocyclic base moiety;
one of R₃′ or R₅′ is Px, wherein Px is a hydroxyl protecting group of formula I, according to claim 1, and the other is selected from:
—P(Pg)(Pn), where Pg is a phosphorus protecting group and Pn is —N(RN1)(RN2), wherein each of RN1 and RN2 is independently selected from hydrogen, substituted or unsubstituted aliphatic, substituted or unsubstituted alicyclic, substituted or unsubstituted aromatic, or substituted or unsubstituted heteroaromatic, or RN1 and RN2 are taken together with the nitrogen atom to which they are attached to form a cyclic moiety selected from substituted or unsubstituted heterocyclic;
-L-ss, where L is a linking moiety and ss is a solid support;
an H-phosphonate moiety; or
a nucleic acid moiety selected from a nucleoside, a nucleotide, a solid support-bound nucleotide, a nucleotide phosphoroamidite, an oligonucleotide, an oligonucleotide blockmer, or a solid support-bound oligonucleotide;
R₂′ is independently selected from OH, alkoxy, substituted alkoxy, halogen, or OPR, where PR is a 2′-protecting group, or a nucleic acid moiety selected from a nucleoside, a nucleotide, a solid support-bound nucleotide, a nucleotide phosphoroamidite, an oligonucleotide, an oligonucleotide blockmer, or a solid support-bound oligonucleotide;
R₄′ is H or R₄′ and R₂′ are taken together to be —(CH₂)ₙ—Y—, where n is 1 or 2 and Y is selected from —O—, —S—, or —N(RN3)-, wherein RN3 is selected from H or substituted or unsubstituted aliphatic; and
R₅′ is selected from H or substituted or unsubstituted alkyl.

13. A compound of claim 12, wherein R₅′ is Px and R₃′ is —P(Pg)(Pn).

14. A compound of claim 13, wherein Pg is —O(CH₂)₂CN and Pn is —N(CH(CH₃)₂)₂.

15. A compound of claim 12, wherein R₂′ is OPR.

16. A compound of claim 15, wherein PR is selected from Px, CPEP, ACE, TOM, TBDMS, or Fpmp.

17. A compound of claim 13, wherein Pn is —N(CH₂CH₃)₂.

18. A compound of claim 17, wherein R₂′ is OPR.

19. A compound of claim 18, wherein PR is CPEP.

20. A compound of claim 12, wherein R₅′ is Px and R₃′ is a nucleic acid moiety selected from a nucleoside, a nucleotide, a solid support-bound nucleotide, a nucleotide phosphoroamidite, an oligonucleotide, an oligonucleotide blockmer, or a solid support-bound oligonucleotide.

21. A compound of claim 12, wherein R₃′ is Px and R₅′ is a nucleic acid moiety selected from a nucleoside, a nucleotide, a solid support-bound nucleotide, a nucleotide phosphoroamidite, an oligonucleotide, an oligonucleotide blockmer, or a solid support-bound oligonucleotide.

22. A compound of claims 20 or 21, wherein any one of said nucleic acid moieties comprises a modified sugar, a 2′substituent, or a conjugate group.

23. A method of synthesizing compounds of formula I, according to claim 1, comprising the steps of:
providing a free hydroxyl of a compound selected from a nucleoside, a nucleotide, a nucleotide phosphoramidite, an oligonucleotide, an oligonucleotide blockmer or a solid support-bound oligonucleotide; and
reacting said compound with a protecting group of formula (III): 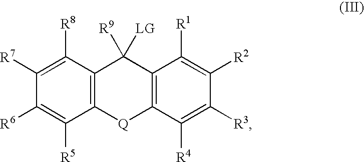
wherein
R¹, R³, R⁴, R⁵, R⁶ and R⁸ are each, independently, H or alkyl or substituted alkyl;
R² and R⁷ are each, independently, alkyl, substituted alkyl, alkenyl, substituted alkenyl, alkynyl, substituted alkynyl, aryl, substituted aryl, hydroxyl, chloro, floro, iodo, cyano, azido, nitro, —C(═O)O—R¹⁰, —O—C(═O)—R¹⁰, —C(═O)N(R¹⁰)R¹¹, —N(R¹⁰)C(═O)R¹¹, —N(R¹⁰)R¹¹, —O—R¹⁰, or —S—R¹⁰;
or two or more groups R¹-R⁸, together with the ring carbons to which they are bonded, combine to form a cyclic moiety selected from substituted or unsubstituted alicyclic, substituted or unsubstituted heterocyclic, substituted or unsubstituted aromatic, or substituted or unsubstituted heteroaromatic;
R⁹ is alkyl, substituted alkyl, alkenyl, substituted alkenyl, alkynyl, substituted alkynyl, aryl or substituted aryl;
R¹⁰ is H or alkyl;
R¹¹ is H or alkyl;
LG is a leaving group; and
Q is O, S, NR¹⁰, or N(C═O)R¹⁰.

24. The method of claim 23, wherein the leaving group is chloro.

25. The method of claim 23, wherein R¹, R³, R⁴, R⁵, R⁶ and R⁸ are each H.
